(19) United States
(12) Patent Application Publication
(10) Pub. No.: US 2022/0243176 A1
(43) Pub. Date: Aug. 4, 2022

(54) SPECIFIC SELECTION OF IMMUNE CELLS USING VERSATILE DISPLAY SCAFFOLDS

(71) Applicants: THE PENN STATE RESEARCH FOUNDATION, University, PA (US); UNIVERSITY OF IOWA RESEARCH FOUNDATION, Iowa City, IA (US)

(72) Inventors: Scott Eugene LINDNER, State College, PA (US); Susan HAFENSTEIN, Petersburg, PA (US); Noah BUTLER, Iowa City, IA (US)

(21) Appl. No.: 17/614,711

(22) PCT Filed: May 20, 2020
(86) PCT No.: PCT/US2020/033785

§ 371 Date: Nov. 29, 2021

Related U.S. Application Data
(60) Provisional application No. 62/855,345, filed on May 31, 2019.

(51) Int. Cl. C12N 5/078

(52) U.S. Cl. CPC C12N 5/0634; B03C 1/02

(57) ABSTRACT

Provided are compositions and methods for use in isolating cells responsive to a target protein by first contacting a collection of isolated cells in an in vitro sample to a complex and then isolating the complex. The complex is formed from a target protein with a capture tag coupled to a multimeric protein structure of at least two self-assembled copies of a monomeric protein substructure fused with a capture sequence.

### CROSS REFERENCE TO RELATED APPLICATIONS

This disclosure claims priority to U.S. Provisional Patent Application 62/855,345 filed May 31, 2019, which is herein incorporated by reference in its entirety.

### STATEMENT OF GOVERNMENT SUPPORT

This invention was made with government support under R01 AI125446 and R01GM125907 awarded by the National Institutes of Health. The government has certain rights in the invention.

### FIELD

The disclosure relates to methods of selecting immune cells from a larger sample and reagents useful for improved selection.

### BACKGROUND

The efficient generation of antibodies with high affinity toward an infectious agent is a hallmark of the immune system. During initial immune responses to an infectious agent or unrecognized antigen, activated naïve B cells form germinal centers that elicit help from T cells to randomly diversify their antibody encoding genes. Clones that exhibit antibodies with higher affinity win the competition for survival within the germinal centers and lead to plasma B cells with long circulation life and memory B cells.

Characterizing B cell responses or isolating B cells with specific antigen recognition has historically been limited to measuring such antibody responses in serum or secretions and sequencing the antibody genes from B cell hybridomas. While many recent advances in the characterization of individual antibody genes from B cell hybridomas has revolutionized the field, they are initially limited by isolation and identification of cells that express the desired receptors for any particular antigen.

As such, new reagents and methods are needed for improved identification of target immune cells.

### SUMMARY

Disclosed are methods of purifying and/or isolating generated immune cells in response to an insult, such as through infection with a virus, parasite or bacterium. The invention provides methods and compositions for use in isolating cells responsive to a target protein by first contacting a collection of isolated cells in an in vitro sample to a complex and then isolating the complex. The complex is formed from a target protein with a capture tag coupled to a multimeric protein structure of at least two self-assembled copies of a monomeric protein substructure fused with a capture sequence. The multimeric protein structure may optionally have a linker and/or a fluorescent protein. Nucleic acids encoding the complex are also included as are kits that include the complex either assembled or as precursors thereto.

The monomeric protein substructure is further fused with a complementary affinity sequence. The complementary affinity sequence can then bind to beads affixed with the complementary binding partner to the complementary affinity sequence, thereby attaching the complex to a solid support. The solid support can be isolated to isolate the complex. For example, the complementary affinity sequence can be biotin and the complementary binding partner be avidin or streptavidin.

The complex can be affixed to a solid support by other approaches as well. The complex can be incubated with a biotinylated antibody that binds to the monomeric protein substructure and then introduced to beads affixed with avidin.

In cases where beads are utilized, the presence of ferromagnetic material in the bead provides a further option to isolate the beads by application of a magnetic field.

The multimeric protein structure is an assembled complex of monomeric protein substructures. The monomeric protein substrucutres can self-assemble to form the multimeric protein structure. The multimeric protein structure features at least two monomeric protein substructures and upwards. In some instances, the multimeric protein structure is made of sixty or more monomeric protein substructures. The monomeric protein substructures may have at least 85% sequence identity with an amino acid selected from the group consisting of SEQ ID NO: 1, SEQ ID NO: 2 or SEQ ID NO: 3.

The monomeric protein substructure may further be fused with capture sequences. The capture sequence binds to a capture tag expressed with a target protein to form the complex. In some instances, the capture sequence has at least 85% sequence identity with an amino acid selected from the group consisting of SEQ ID NO: 7, SEQ ID NO: 8 or SEQ ID NO: 9.

The monomeric protein substructure is further fused with a fluorophore to render the complex visible and also provide a further mechanism for isolating cells associated with the complex, such as by flow cytometry. The monomeric protein substructure may have at least 85% sequence identity with an amino acid selected from the group consisting of SEQ ID NO: 16, SEQ ID NO: 17, SEQ ID NO: 18 or SEQ ID NO: 19.

The target protein may be fused with the capture tag that binds the capture sequence to assemble the complex. The capture tag may have at least 90% sequence identity with an amino acid selected from the group consisting of SEQ ID NO: 26 or SEQ ID NO: 27.

The target protein of the complex can associate with cells in vitro and the subsequent isolation of cells allows for identification of cells that recognize the target protein. In some instances, the collection of cells can include adaptive immune cells, such as B cells and/or T cells. Isolation of the complex therefor allows for identification of adaptive immune cells that specifically recognize the target protein.

Cells isolated by the complex may be further processed. For example, isolated cells can further be placed an in vitro cell culture or harvested to identify particular nucleic acids, such as to isolate a nucleic acid encoding an antibody. Nucleic acids encoding an antibody can be then inserted into an expression vector.

In some instances, a second complex can be incubated with the collection of cells. This second complex can feature a second target protein different from the first, such as a decoy or a negative control protein for the target protein. The second complex can help to confirm binding specificity to the first complex.

The methods and compositions may further provide for assaying a subject for immunity to a target protein by incubating a collection of cells from the subject with the complex.

The methods and compositions may further provide for preparing a B cell in vitro tissue culture with binding affinity to the target protein of the complex. Following isolation of the complex, B cells can be isolated from the complex and transferred to a tissue culture medium.

### BRIEF DESCRIPTION OF THE DRAWINGS

FIG. 1 shows a stained 12% SDS-PAGE gel demonstrating the successful expression and isolation of a multimeric construct according to some aspects as described herein with red fluorescent protein fused to each monomer of the capture scaffold.

FIG. 2 shows a stained 12% SDS-PAGE gel demonstrating the successful expression and isolation of a biotinylated, multimeric construct according to some aspects as described herein with red, green or blue fluorescent proteins fused to each monomer of the capture scaffold.

FIG. 3 shows a western blot probed with streptavidin-HRP of biotinylated, multimeric constructs according to some aspects as described herein to detect the presence of biotin associated with the constructs. In all three fluorescent protein variants, biotinylation was confirmed.

FIG. 4 shows a 10% SDS-PAGE gel confirming successful association between unbiotinylated multimeric protein structure according to some aspects as described herein and exemplary target proteins. The upper left arrow/bracket confirms covalent bonding between MSP1(19) or UIS4 with the multimeric protein structure, with unbonded MSP1(19) indicated by the lower left arrow and unbonded UIS4 indicated by the lower right arrow. The upper right arrow highlights that not all of the capture scaffold bonded with MSP1(19). A PageRuler Plus pre-stained ladder was used to confirm protein mobility and approximate molecular weight.

FIG. 5 shows a western blot probed with purified IgG raised against an exemplary multimeric protein structure constructs as provided herein as the primary antibody and goat anti-rabbit IgG-HRP as the secondary antibody to confirm production and isolation of antibodies to the monomeric structures. The two lanes were loaded with 100 ng and 10 ng of multimeric protein structure constructs, going from left to right.

FIG. 6 shows a western blot probed using streptavidin-HRP to confirm that both heavy chain and light chain of an antibody raised against the multimeric protein structure constructs are successfully biotinylated in vitro by a chemical crosslinker according to some aspects as provided herein.

FIG. 7A shows a schematic overview of the method for isolating B cells according to some aspects as provided herein using either biotinylated or the unbiotinylated variants of an exemplary multimeric protein structure construct illustrating that the biotinylated multimeric protein structure construct can be coupled to streptavidin-coated beads immediately following incubation, while the unbiotinylated variant is incubated with biotinylated antibodies to the capture scaffold protein first.

FIG. 7B shows a schematic overview of an exemplary method for isolating B cells according to some aspects as provided herein using either the biotinylated or the unbiotinylated variants of an exemplary multimeric protein structure construct illustrating that following capture of B cells with the beads (thereby selecting the positive fraction), the assembled complex can be resolved by FACS, with gating options to identify those complexes that are antigen specific.

FIG. 8A shows the results following FACS from the positive fractions obtained from application of a magnetic field to retain the complexes using the unbiotinylated multimeric protein structure construct. The left panels in each validate that the bounds cells are B cells. The right panels represent an alternative strategy designed to confirm the bounds cells are B cells.

FIG. 8B shows the results following FACS from the negative fractions multimeric protein structure from application of a magnetic field to retain the complexes using the unbiotinylated multimeric protein structure construct.

FIG. 9 shows FACS data for B cell isolation in naive (lower) and P. yoelii inoculated (upper) mice. The boxed regions show the successful identification of MSP1(19) specific B cells by unbiotinylated multimeric protein structure constructs according to some aspects as provided herein.

FIG. 10 shows MSP1(19)-specific B cell isolation with the biotinylated multimeric protein structure constructs in P. yoelii inoculated mice as compared to naïve mice. These data show success of the biotinylated multimeric protein structure constructs in identifying B cells specific to P. yoelii MSP1(19).

FIG. 11 shows a comparison between the tetramer system and biotinylated multimeric protein structure constructs according to some aspects as provided herein. The FACS data show that the biotinylated variant outperforms the tetramer model in identifying B-cells that bind specifically to PyMSP1(19).

### DETAILED DESCRIPTION

Provided are processes and reagents that have utility for improved recognition of target cells such as immune cells. The processes capitalize on improved large and rigid protein structures designed to be capable of efficiently and rapidly expressing any desired target antigen, antibody, or other molecule. These systems can also express specific labels (e.g. fluorophores, genetically encoded fluorescent proteins) that emit far more signal than prior systems thereby allowing efficient recognition of even low quantity target cells.

The processes of recognizing and optionally isolating a target immune cell as provided herein utilizes a self-assembling multimeric protein structure (optionally non-naturally occurring) to form a target complex and binding that target complex to one or more target cells within a mixed population of cells to identify and optionally isolate the target cells. The self-assembling multimeric protein structures as provided herein and used for structural biology applications, may in some aspects display up to 60 copies of the same antigen or antibody protein onto the cage sphere. Further associating one or more fluorophores with the cage proteins allows for 10-fold increases in fluorescence intensities for identification and isolation by methods such as fluorescence-activated cell sorting (FACS). By binding specific agents capable of recognizing magnetic beads or other recognition units designed for purification and enrichment, the system may be used for binding target cells to magnetic beads and subsequent isolation by magnetic-activated cell sorting (MACS) or other such methods.

### Multimeric Protein Structure

A multimeric protein structure as provided herein is a multimer of smaller proteins that assemble, optionally without the aid of external stimuli (self-assembling) to form the multimeric protein structure, optionally termed a “nanocage” or “multimeric construct” in this disclosure. In some figures and construct names, the multimeric protein structure may be called “cage” or “capture scaffold” for brevity purposes. The smaller proteins are optionally protein substructures. The multimeric protein structure construct is the result of union of the monomer protein substructures into a substantially rigid multimeric assembly.

A “protein” as used herein is an assembly of two or more amino acids linked by a peptide bond.

An “antigen” as used herein is a protein that is capable of eliciting an immune response in a subject either alone or with the aid of one or more adjuvants.

The plurality of protein substructures self-assemble to form the multimeric protein structure construct (cage). As is recognized in the art, self-assembly is the oligomerization of protein substructures into an ordered arrangement driven by non-covalent interactions. Such non-covalent interactions may be any of electrostatic interactions, π-interactions, van der Walls forces, hydrogen bonding, hydrophobic effects, or any combination thereof. The resulting multimeric protein structure is optionally ordered into a shape, illustratively an icosahedron, but other shapes may be used as well for example those with symmetry including trimeric, tetrahedral, octahedral, or dodecahedral. Illustrative examples of such multimeric protein structures and how to make them are illustrated in WO 2016/138525, WO 2018/170362, and U.S. Patent Application Publication No: 2015/0356240.

The number of protein substructures in an assembled multimeric protein structure is dependent on the overall arrangement. In some aspects, the number of protein substructures is 60 forming an icosahedron, however other structures with different numbers of substructures are similarly useful such as 24 protein subunit structures illustratively as that described by King, et al., Nature, 510, 103-108 (2014), or 12 protein subunit structures such as that described by King, et al., Science, 336, 1171-1174 (2012), 4-protein subunit structures illustratively as that described by Liu et al., PNAS, March 27, 2018 115 (13) 3362-3367.

It is appreciated that in some aspects all protein substructures may be identical in primary sequence thereby promoting identity in structure to form a homo-multimeric protein structure. However, there may be some structures where two or more different protein substructures are used. Optionally, 2, 3, 4, 5, or more different monomer protein substructures may be used to form the multimeric protein structure.

Optionally, the monomer protein substructures are forms of aldolase protein, optionally structurally modified so as to either alter self-assembly properties, increase rigidity of the final multimeric protein structure, to express one or more tags for purification, to express one or more tags for associating with a target protein or combinations thereof. In some aspects, the protein substructures are one or more of those described by Hsia, et al., Nature, 2016; 535:136-147 or those designed and described in WO 2016/138525A1 with either optionally modified otherwise as described herein.

Optionally, a monomer protein substructure includes the primary sequence as defined in

SEQ ID NO: 1
(MEELFKKHKIVAVLRANSVEEAKKKALAVFLGGVH
LIEITFTVPDADTVIKELSFLKEMGAIIGAGTVTS
VEQCRKAVESGAEFIVSPHLDEEISQFCKEKGVFY
MPGVMTPTELVKAMKLGHTILKLFPGEVVGPQFVK
AMKGPFPNVKFVPTGGVNLDNVCEWFKAGVLAVGV
GSALVKGTPVEVAEKAKAFVEKIRGCTEHM),
optionally SEQ ID NO: 2
(MEELFKKHKIVAVLRANSVEEAKKKALAVFLGGVH
LIEITFTVPDADTVIKELSFLKEMGAIIGAGTVTS
VEQCRKAVESGAEFIVSPHLDEEISQFCKEKGVFY
MPGVMTPTELVKAMKLGHTILKLFPGEVVGPQFVK
AMKGPFPNVKFVPTGGVNLDNVCEWFKAGVLAVGV
GSALVKGTPVEVAEKAKAFVEKIRGCTEHM),
optionally SEQ ID NO: 3
(FKKHKIVAVLRANSVEEAKKKALAVFLGGVHLIEI
TFTVPDADTVIKELSFLKEMGAIIGAGTVTSVEQC
RKAVESGAEFIVSPHLDEEISQFCKEKGVFYMPGV
MTPTELVKAMKLGHTILKLFPGEVVGPQFVKAMKG
PFPNVKFVPTGGVNLDNVCEWFKAGVLAVGVGSAL
VKGTPVEVAEKAKAFVEKIRGCTEHM)

In some aspects, a monomer protein substructure further includes additional residues at an N or C terminus that may be due to translations from endonuclease restriction sites, tags such as for purification (e.g. 6xHis tag), a specific protease cleavage site such as a thrombin cleavage site, or other suitable modification. In some aspects, the monomer protein substructures include the primary sequence of

SEQ ID NO: 4
(MKMEELFKKHKIVAVLRANSVEEAKKKALAVFLGG
VHLIEITFTVPDADTVIKELSFLKEMGAIIGAGTV
TSVEQCRKAVESGAEFIVSPHLDEEISQFCKEKGV
FYMPGVMTPTELVKAMKLGHTILKLFPGEVVGPQF
VKAMKGPFPNVKFVPTGGVNLDNVCEWFKAGVLAV
GVGSALVKGTPVEVAEKAKAFVEKIRGCTEHM),
SEQ ID NO: 5
(ASMEELFKKHKIVAVLRANSVEEAKKKALAVFLGG
VHLIEITFTVPDADTVIKELSFLKEMGAIIGAGTV
TSVEQCRKAVESGAEFIVSPHLDEEISQFCKEKGV
FYMPGVMTPTELVKAMKLGHTILKLFPGEVVGPQF
VKAMKGPFPNVKFVPTGGVNLDNVCEWFKAGVLAV
GVGSALVKGTPVEVAEKAKAFVEKIRGCTEHM)
or
SEQ ID NO: 6
(EELFKKHKIVAVLRANSVEEAKKKALAVFLGGVHL
IEITFTVPDADTVIKELSFLKEMGAIIGAGTVTSV
EQCRKAVESGAEFIVSPHLDEEISQFCKEKGVFYM
PGVMTPTELVKAMKLGHTILKLFPGEVVGPQFVKA
MKGPFPNVKFVPTGGVNLDNVCEWFKAGVLAVGVG
SALVKGTPVEVAEKAKAFVEKIRGCTERM).

The monomer protein substructures are optionally modified at one or more amino acid positions relative to any one or more of SEQ ID Nos: 1-6 or others as provided herein. Optionally, the protein substructures are 70% identical or greater to any one or more of those provided herein, optionally 75% or more identical, optionally 80% or more identical, optionally 85% or more identical, optionally 90% or more identical, optionally 95% or more identical, optionally 96% or more identical, optionally 97% or more identical, optionally 98% or more identical, optionally 99% or more identical. Illustrative residues that may be substituted include E26 optionally substituted to K, E33 optionally substituted to L, K61 optionally substituted to M, D187 optionally substituted to V and R190 optionally substituted to A, in one or more of SEQ ID Nos 1-6. Optionally, other substitutions may be made such as deletion of any of the first 10 residues at the N- or C-termini of the protein substructures. In some aspects, an extra M is added to the N-terminus so as to extend the alpha helical structure, optionally into an alpha helical linker.

Modifications and changes can be made in the structure of the monomer protein substructure primary sequences that are the subject of the application and still obtain a molecule having similar characteristics as the original such as similar self-assembly properties, similar rigidity to the final multimeric protein structure, or other. Such substitutions are optionally conservative amino acid substitutions. For example, certain amino acids can be substituted for other amino acids in a sequence without appreciable alteration of desired properties. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 are optional, those within ±1 are optional preferred, and those within ±0.5 are optional.

Substitution of like amino acids can also be made on the basis of hydrophilicity, particularly, where the biological functional equivalent polypeptide or peptide thereby created is intended for use in particular aspects as described herein. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Aspects of this disclosure thus contemplate functional or biological equivalents of a polypeptide as set forth above. In particular, aspects of the polypeptides can include variants having about 50%, 60%, 70%, 80%, 90%, and 95% sequence identity to the polypeptide of interest.

One or more of the protein substructures is optionally modified at the N-terminus, the C-terminus or both with one or more of a linker, a capture sequence, a fluorescent protein, recognition unit (e.g. antibody or other capable of binding a magnetic bead or other purification or identification component), or combinations thereof. One power of the substructures as provided herein is the ability to create self-assembling multimeric protein structures that express capture sequences oriented either out and away from the multimeric protein structure such as through an N-terminal capture sequence, directed into the core of the multimeric protein structure such as through a C-terminal capture sequence or both. A capture sequence may be located in any position of the protein, including directly at the N- or C-terminus, in flexible loop regions of the protein structure, within between about 10 and 30 amino acids from the N- or C-terminus, optionally in substitution of or within 10 amino acids of the N- or C-terminus of any one or more of SEQ ID Nos: 1-6.

One advantage of a capture sequence is that it eliminates the need for genetic fusions of target proteins-of-interest with the self-assembling multimeric protein structure. For example, prior preparations used as a label required that the monomer protein substructures be recombinantly expressed already fused to the target protein-of-interest, increasing complexity of making the materials as well as reducing the likelihood of success. Moreover, if the protein-of-interest is optimally expressed in a cell type other than bacteria (e.g. yeast, insect cells, mammalian cells) to add appropriate post-translational modifications, this capture scaffold allows for this constraint. The use of a capture sequence that can pair with a capture tag sequence on a target protein-of-interest increases the robustness of the resulting multimeric protein structure, but also allows for adjustment of parameters such as saturation of target protein on the multimeric protein structure that were found to improve the resulting functional aspects of the multimeric protein structures.

As such, a monomer protein substructure optionally includes one or more capture sequences. Illustrative examples of a capture sequence include those that allow specific recognition of the capture sequence by the capture tag on the target protein and lead to covalent bonding of the two, optionally through the use of a spontaneous isopeptide bond. Optionally, a capture sequence terminates with an alkylamine or other functional group that can pair with a capture tag on a target protein's sequence. Optionally, the capture tag on the target protein's sequence terminates in a carboxylic acid allowing isopeptide bond formation with the capture sequence. This results in robust covalent bonding between the multimeric protein structure (nanocage) and the target protein of interest. As set forth in the examples described herein, a capture sequence allows for a desired capture tagged target protein to associate with the multimeric protein structure when expressed to form a complex. The strength of the bond between the target protein's capture tag and the capture sequence allows for subsequent isolation of B and/or T cells that recognize the target protein via their association with the complex.

In some aspects, a capture sequence is or includes biotin, avidin,

SEQ ID NO: 7
(GSGDSATHIKFSKRDEDGKELAGATMELRDSSGKT
ISTWISDGQVKDFYLYPGKYTFVETAAPDGYEVAT
AITFTVNEQGQVTVNGKATKGDAHIGVD),
SEQ ID NO: 8
(MGSSHEIHHHHGSGDSATHIKFSKRDEDGKELAGA
TMELRDSSGKTISTWISDGQVKDFYLYPGKYTFVE
TAAPDGYEVATAITFTVNEQGQVTVNGKATKGDAH
IGVD),
SEQ ID NO: 9
(MKPLRGAVFSLQKQHPDYPDIYGAIDQNGTYQNVR
TGEDGKLTFKNLSDGKYRLFENSEPAGYKPVQNKP
IVAFQIVNGEVRDVTSIVPQDIPATYEFTNGKHYI
TNEPIPPK),

any functional portion thereof, a nucleic acid (e.g., deoxyribonucleic acid, or ribonucleic acid) sequence, or other such suitable capture sequence, or any combination thereof. A suitable capture sequence is one that will bind, either covalently or non-covalently, and specifically with a capture tag or other desired portion of a target molecule.

In some aspects one or more monomer protein substructures of a multimeric protein structure includes a linker, the linker bound to the protein substructure and the capture sequence, optionally between the protein substructure and the capture sequence. The linker optionally covalently or non-covalently (e.g. hydrogen bonding, van der Walls forces, hydrophobic effects, electrostatic interactions, π-interactions, or combinations thereof), or both, binds the monomer protein substructure to the capture sequence.

A linker is optionally a protein linker, single amino acid, nucleic acid based linker such as one or more nucleotides (e.g., ribonucleotides, deoxyribonucleotide), a nucleic acid of two or more nucleotides, a substituted or unsubstituted alkyl, akenyl, or alkynyl of 1-20 carbons, or other suitable structure. Optionally, a linker is a flexible linker or a rigid linker. A flexible linker is one that is not restricted by interlinker bonding or regular three dimensional structure in an aqueous environment at 25° C. A rigid linker is one that includes one or more interlinker bonds (either covalent or non-covalent) (e.g. electrostatic interaction, disulfide bond, or other) or forms a secondary structure (e.g. alpha helix, beta sheet, beta turn, omega loop) that is stable in an aqueous environment at 25° C.

Optionally, a linker is a protein linker of two or more amino acids. Illustrative protein linkers include, but are not limited to one or more multimers of the sequence GGS, GSS, PPA, EAAAK (SEQ ID NO: 10), a proline residue, or combinations thereof. A multimer of any of the forgoing optionally include 2, 3, 4, 5, 6, 7, 8, 9, or more repeats or substitutions of the foregoing. In specific examples, a linker has a sequence of 5 repeats of GGS, 5 repeats of GSS, 5 or more linked GGS and GSS sequences in any order, 5 repeats of SEQ ID NO: 10, a 9-mer of proline residues, a 3-mer of the sequence PPA, or any combination thereof.

As such, a monomer protein substructure optionally includes a self-assembling monomer protein, a linker, and a capture sequence where the linker and the capture sequence are optionally bound to the self-assembling monomer at the N-terminus, the C-terminus, or both. Illustrative examples of protein substructures include but are not limited to those of SEQ ID NO: 11

SEQ ID NO: 11
(MGSSHEIHHHHGSGDSATHIKFSKRDEDGKELAGA
TMELRDSSGKTISTWISDGQVKDFYLYPGKYTFVE
TAAPDGYEVATAITFTVNEQGQVTVNGKATKGDAH
IGVDHEIHHHHGGSGGSGGSGGSMKMEELFKKHKI
VAVLRANSVEEAKKKALAVFLGGVHLIEITFTVPD
ADTVIKELSFLKEMGAIIGAGTVTSVEQCRKAVES
GAEFIVSPHLDEETSQFCKEKGVFYMPGVMTPTEL
VKAMKLGHTILKLFPGEVVGPQFVKAMKGPFPNVK
FVPTGGVNLDNVCEWFKAGVLAVGVGSALVKGTPV
EVAEKAKAFVEKIRGCTERM),
SEQ ID NO: 12
(MGSSHEIHHHHGSGDSATHIKFSKRDEDGKELAGA
TMELRDSSGKTISTWISDGQVKDFYLYPGKYTFVE
TAAPDGYEVATAITFTVNEQGQVTVNGKATKGDAH
IGVDEAAAKEAAAKEAAAKEAAAKEAAAKASMEEL
FKKHKIVAVLRANSVEEAKKKALAVFLGGVHLIEI
TFTVPDADTVIKELSFLKEMGAIIGAGTVTSVEQC
RKAVESGAEFIVSPHLDEEISQFCKEKGVFYMPGV
MTPTELVKAMKLGHTILKLFPGEVVGPQFVKAMKG
PFPNVKFVPTGGVNLDNVCEWFKAGVLAVGVGSAL
VKGTPVEVAEKAKAFVEKIRGCTERM),
SEQ ID NO: 13
(MGSSHEIHHHHGSGDSATHIKFSKRDEDGKELAGA
TMELRDSSGKTISTWISDGQVKDFYLYPGKYTFVE
TAAPDGYEVATAITFTVNEQGQVTVNGKATKGDAH
IGVDEAAAKEAAAKEAAAKEAAAKEAAAKEELFKK
HKIVAVLRANSVEEAKKKALAVFLGGVHLIEITFT
VPDADTVIKELSFLKEMGAIIGAGTVTSVEQCRKA
VESGAEFIVSPHLDEEISQFCKEKGVFYMPGVMTP
TELVKAMKLGHTILKLFPGEVVGPQFVKAMKGPFP
NVKFVPTGGVNLDNVCEWFKAGVLAVGVGSALVKG
TPVEVAEKAKAFVEKIRGCTEHM),
SEQ ID NO: 14
(MGSSHEIHHHEGSGDSATHIKFSKRDEDGKELAGA
TMELRDSSGKTISTWISDGQVKDFYLYPGKYTFVE
TAAPDGYEVATAITFTVNEQGQVTVNGKATKGDAH
IGVDPPPPPPPPPEELFKKHKIVAVLRANSVEEAK
KKALAVFLGGVHLIEITFTVPDADTVIKELSFLKE
MGAIIGAGTVTSVEQCRKAVESGAEFIVSPHLDEE
ISQFCKEKGVFYMPGVMTPTELVKAMKLGHTILKL
FPGEVVGPQFVKAMKGPFPNVKFVPTGGVNLDNVC
EWFKAGVLAVGVGSALVKGTPVEVAEKAKAFVEKI
RGCTEHM),
or
SEQ ID NO: 15
(MGSSHEIHHHEGSGDSATHIKFSKRDEDGKELAGA
TMELRDSSGKTISTWISDGQVKDFYLYPGKYTFVE
TAAPDGYEVATAITFTVNEQGQVTVNGKATKGDAH
IGVDPPAPPAPPAEELFKKHKIVAVLRANSVEEAK
KKALAVFLGGVHLIEITFTVPDADTVIKELSFLKE
MGAIIGAGTVTSVEQCRKAVESGAEFIVSPHLDEE
ISQFCKEKGVFYMPGVMTPTELVKAMKLGHTILKL
FPGEVVGPQFVKAMKGPFPNVKFVPTGGVNLDNVC
EWFKAGVLAVGVGSALVKGTPVEVAEKAKAFVEKI
RGCTERM).

In some aspects, one or more monomer protein substructures of a self-assembling multimeric protein structure optionally include a complementary affinity sequence expressed as part of the multimeric protein structure. Such sequences may be bound directly or indirectly to the monomer protein substructure and/or the capture sequence, optionally spaced apart by a linker. In some instances, the sequence is recognized and modified by a ligase, such as E. coli BirA. The complementary affinity sequence may be found at any position in the protein, including at either terminus of the multimeric protein structure or within up to 10 amino acids of a terminus (e.g. SEQ ID NO: 38). As with a capture sequence, a complementary affinity sequence pairs with a complementary binding partner. A complementary affinity sequence may comprise a second capture sequence within the multimeric protein structure.

The complementary affinity sequence may provide a further option for use in isolating associated immune cells based on its affinity to its complementary binding partner. Complementary in this sense means that the complementary affinity sequence will bind to, optionally specifically bind to, its complementary binding partner sequence, optionally with high affinity. In some instances, the complementary affinity sequence is a biotin group, peptide that can bind to biotin, or a multimeric or monomeric streptavidin or avidin sequence. As used herein, when biotin is utilized as the complementary affinity sequence, multimeric protein structures are referred to as biotinylated variants (or biotin cage for brevity in some construct names or figure descriptions). Similarly, a multimeric protein structure lacking a biotin affinity sequence may be referred to as unbiotinylated.

In instances such as where biotin or avidin are already utilized as capture sequences, other complementary affinity interactions can be utilized in the expressed multimeric protein structure, such as that seen between the complementary affinity sequence of SEQ ID NO: 26 and its complementary binding partner SEQ ID NO: 7 or complementary affinity sequence of SEQ ID NO: 27 and its complementary binding partner SEQ ID NO: 9.

While a capture sequence is to append a target protein to the multimeric protein structure as discussed herein and subsequently attract a B and/or T cell to the expressed complex, the relationship between the complementary affinity sequence and its complementary binding partner allows for additional purification steps, such as direct coupling to a solid support. By way of example, the complementary binding partner of the complementary affinity sequence can be affixed to a solid support. As a result, the complementary affinity sequence can couple the multimeric protein structure to the solid support via the binding affinity of the complementary pair. For example, expression of biotin as a complementary affinity sequence allows for a strong interaction with streptavidin or avidin as its complementary binding partner, which when coupled to a solid support, allows the entire complex and proteins associated therewith to be isolated from a mixed lysate or similar.

In some instances, a complementary affinity sequence can be appended by inserting a DNA sequence for each monomer protein substructure of the multimeric protein structure in an open reading frame of an expression vector that includes such. In other instances, a complementary affinity sequence may be ligated to a monomer protein substructure. As a specific example, a biotin tag may be introduced by ligation with the naturally occurring protein sequence recognized by the E. coli Bir A biotin ligase enzyme.

The complementary binding partner is a protein or active peptide fragment with specific binding to the complementary affinity sequence. The complementary binding partner is fused to a solid support, optionally by a linker. When fused to the solid support, the complementary binding partner retains sufficient structure such that its ability to specifically bind the complementary affinity sequence is not impaired. A linker or tether may be utilized to affix the complementary binding partner to a solid support to ensure binding affinity remains. The attachment to a solid support of the complementary binding partner allows for the entire assembled multimeric protein structure to be isolated straightforwardly. When the capture tag is engaged with the capture sequence as discussed herein, the target protein is also capable of being isolated. The solid support can be isolated, for instance by gravity or centrifugation. In instances where the solid support is ferromagnetic, application of a magnetic field can be utilized.

In some particular aspects as provided herein a monomer protein substructure optionally includes: a self-assembling monomer protein; a linker at the N-terminus, C-terminus or both; one or more capture sequence at the N-terminus, C-terminus or both; and a fluorescent protein at the N-terminus, C-terminus or both. Other protein substructures optionally include: a self-assembling monomer protein; a linker at the N-terminus, C-terminus or both; a capture sequence at or proximal, with respect to the self-assembling monomer protein, to the N-terminus, C-terminus or both; a complementary affinity sequence at or proximal to the N-terminus, C-terminus or both; and a detection label such as a fluorescent protein, radiolabel or similar at or proximal to the N-terminus, C-terminus or both. A fluorescent protein optionally emits in the green, red, or blue regions of the visible spectrum. Optionally, a fluorescent protein is a known fluorescent protein such as mScarlet (Bindels, et al., Nature Methods, volume 14, pages 53-56 (2017)), mNeonGreen (Shaner, et al., Nature Methods, 2013 May; 10(5): 407-409), mTurquoise2 (Geodhart, et al., Nat Commun. 2012 Mar. 20; 3: 751), or others as recognized in the art. Specific illustrative examples of protein substructures that may or may not further include a fluorescent protein on the C-terminus as provided herein may be or include amino acid sequences as follows:

| | |
|---|---|
| Capture-Cage-Red | MGSSHHHHHHGSGDSATHIKFSKRD |
| SEQ ID NO: 16 | EDGKELAGATMELRDSSGKTISTWI |
| (unbiotinylated) | SDGQVKDFYLYPGKYTFVETAAPDG |
| | YEVATAITFTVNEQGQVTVNGKATK |
| | GDAHIGVDHHHHHHGGSGGSGGSGG |
| | SMKMEELFKKHKIVAVLRANSVEEA |
| | KKKALAVFLGGVHLIEITFTVPDAD |
| | TVIKELSFLKEMGAIIGAGTVTSVE |
| | QCRKAVESGAEFIVSPHLDEEISQF |
| | CKEKGVFYMPGVMTPTELVKAMKLG |
| | HTILKLFPGEVVGPQFVKAMKGPFP |
| | NVKFVPTGGVNLDNVCEWFKAGVLA |
| | VGVGSALVKGTPVEVAEKAKAFVEK |
| | IRGCTEHMGGSGGSGGSGGSVSKGE |
| | AVIKEFMRFKVHMEGSMNGHEFEIE |
| | GEGEGRPYEGTQTAKLKVTKGGPLP |
| | FSWDILSPQFMYGSRAFTKHPADIP |
| | DYYKQSFPEGFKWERVMNFEDGGAV |
| | TVTQDTSLEDGTLIYKVKLRGTNFP |
| | PDGPVMQKKTMGWEASTERLYPEDG |
| | VLKGDIKMALRLKDGGRYLADFKTT |
| | YKAKKPVQMPGAYNVDRKLDITSHN |
| | EDYTVVEQYERSEGRHSTGGMDELY |
| | K |
| Capture-Cage- | MGSSHHHHHHGSGDSATHIKFSKRD |
| Green | EDGKELAGATMELRDSSGKTISTWI |
| SEQ ID NO: 17 | SDGQVKDFYLYPGKYTFVETAAPDG |
| (unbiotinylated) | YEVATAITFTVNEQGQVTVNGKATK |
| | GDAHIGVDHHHHHHGGSGGSGGSGG |
| | SMKMEELFKKHKIVAVLRANSVEEA |
| | KKKALAVFLGGVHLIEITFTVPDAD |
| | TVIKELSFLKEMGAIIGAGTVTSVE |
| | QCRKAVESGAEFIVSPHLDEEISQF |
| | CKEKGVFYMPGVMTPTELVKAMKLG |
| | HTILKLFPGEVVGPQFVKAMKGPFP |
| | NVKFVPTGGVNLDNVCEWFKAGVLA |
| | VGVGSALVKGTPVEVAEKAKAFVEK |
| | IRGCTEHMGGSGGSGGSGGSMVSKG |
| | EEDNMASLPATHELHIFGSINGVDF |
| | DMVGQGTGNPNDGYEELNLKSTKGD |
| | LQFSPWILVPHIGYGFHQYLPYPDG |
| | MSPFQAAMVDGSGYQVHRTMQFEDG |
| | ASLTVNYRYTYEGSHIKGEAQVKGT |
| | GFPADGPVMTNSLTAADWCRSKKTY |
| | PNDKTIISTFKWSYTTGNGKRYRST |
| | ARTTYTFAKPMAANYLKNQPMYVFR |
| | KTELKHSKTELNFKEWQKAFTDVMG |
| | MDELYK |
| BiotynCage-Red | MGLNDIFEAQKIEWHEGGSGGSGGS |
| SEQ ID NO: 18 | HHHHHHGSGDSATHIKFSKRDEDGK |
| (biotinylated) | ELAGATMELRDSSGKTISTWISDGQ |
| | VKDFYLYPGKYTFVETAAPDGYEVA |
| | TAITFTVNEQGQVTVNGKATKGDAH |
| | IGVDGGSGGSGGSGGSMKMEELFKK |
| | HKIVAVLRANSVEEAKKKALAVFLG |
| | GVHLIEITFTVPDADTVIKELSFLK |
| | EMGAIIGAGTVTSVEQCRKAVESGA |
| | EFIVSPHLDEEISQFCKEKGVFYMP |
| | GVMTPTELVKAMKLGHTILKLFPGE |
| | VVGPQFVKAMKGPFPNVKFVPTGGV |
| | NLDNVCEWFKAGVLAVGVGSALVKG |
| | TPVEVAEKAKAFVEKIRGCTEHMGG |
| | SGGSGGSGGSVSKGEAVIKEFMRFK |
| | VHMEGSMNGHEFEIEGEGEGRPYEG |
| | TQTAKLKVTKGGPLPFSWDILSPQF |
| | MYGSRAFTKHPADIPDYYKQSFPEG |
| | FKWERVMNFEDGGAVTVTQDTSLED |
| | GTLIYKVKLRGTNFPPDGPVMQKKT |
| | MGWEASTERLYPEDGVLKGDIKMAL |
| | RLKDGGRYLADFKTTYKAKKPVQMP |
| | GAYNVDRKLDITSHNEDYTVVEQYE |
| | RSEGRHSTGGMDELYK |
| BiotynCage- | MGLNDIFEAQKIEWHEGGSGGSGGS |
| Green | HHHHHHGSGDSATHIKFSKRDEDGK |
| SEQ ID NO: 19 | ELAGATMELRDSSGKTISTWISDGQ |
| (biotinylated) | VKDFYLYPGKYTFVETAAPDGYEVA |
| | TAITFTVNEQGQVTVNGKATKGDAH |
| | IGVDGGSGGSGGSGGSMKMEELFKK |
| | HKIVAVLRANSVEEAKKKALAVFLG |
| | GVHLIEITFTVPDADTVIKELSFLK |
| | EMGAIIGAGTVTSVEQCRKAVESGA |
| | EFIVSPHLDEEISQFCKEKGVFYMP |
| | GVMTPTELVKAMKLGHTILKLFPGE |
| | VVGPQFVKAMKGPFPNVKFVPTGGV |
| | NLDNVCEWFKAGVLAVGVGSALVKG |
| | TPVEVAEKAKAFVEKIRGCTEHMGG |
| | SGGSGGSGGSMVSKGEEDNMASLPA |
| | THELHIFGSINGVDFDMVGQGTGNP |
| | NDGYEELNLKSTKGDLQFSPWILVP |
| | HIGYGFHQYLPYPDGMSPFQAAMVD |
| | GSGYQVHRTMQFEDGASLTVNYRYT |
| | YEGSHIKGEAQVKGTGFPADGPVMT |
| | NSLTAADWCRSKKTYPNDKTIISTF |
| | KWSYTTGNGKRYRSTARTTYTFAKP |
| | MAANYLKNQPMYVFRKTELKHSKTE |
| | LNFKEWQKAFTDVMGMDELYK |
| BiotynCage-Blue | MGLNDIFEAQKIEWHEGGSGGSGGS |
| SEQ ID NO: 20 | HHHHHHGSGDSATHIKFSKRDEDGK |
| (biotinylated) | ELAGATMELRDSSGKTISTWISDGQ |
| | VKDFYLYPGKYTFVETAAPDGYEVA |
| | TAITFTVNEQGQVTVNGKATKGDAH |
| | IGVDGGSGGSGGSGGSMKMEELFKK |
| | HKIVAVLRANSVEEAKKKALAVFLG |
| | GVHLIEITFTVPDADTVIKELSFLK |
| | EMGAIIGAGTVTSVEQCRKAVESGA |
| | EFIVSPHLDEEISQFCKEKGVFYMP |
| | GVMTPTELVKAMKLGHTILKLFPGE |
| | VVGPQFVKAMKGPFPNVKFVPTGGV |
| | NLDNVCEWFKAGVLAVGVGSALVKG |
| | TPVEVAEKAKAFVEKIRGCTEHMGG |
| | SGGSGGSGGSMVSKGEELFTGVVPI |
| | LVELDGDVNGHKFSVSGEGEGDATY |
| | GKLTLKFICTTGKLPVPWPTLVTTL |
| | SWGVQCFARYPDHMKQHDFFKSAMP |
| | EGYVQERTIFFKDDGNYKTRAEVKF |
| | EGDTLVNRIELKGIDFKEDGNILGH |
| | KLEYNYFSDNVYITADKQKNGIKAN |
| | FKIRHNIEDGGVQLADHYQQNTPIG |
| | DGPVLLPDNHYLSTQSKLSKDPNEK |
| | RDHMVLLEFVTAAGITLGMDELYK |

Specific illustrative examples of nucleotide sequences that may be used to express one or more of the above amino acid sequences including a fluorescent protein may be as follows:

| | |
|---|---|
| Capture-Cage-Red | ATGGGCAGCAGCCATCATCATCATC |
| SEQ ID NO: 21 | ATCACGGCAGCGGCGATAGTGCTAC |
| | CCATATTAAATTCTCAAAACGTGAT |
| | GAGGACGGCAAAGAGTTAGCTGGTG |
| | CAACTATGGAGTTGCGTGATTCATC |
| | TGGTAAAACTATTAGTACATGGATT |
| | TCAGATGGACAAGTGAAAGATTTCT |
| | ACCTGTATCCAGGAAAATATACATT |
| | TGTCGAAACCGCAGCACCAGACGGT |
| | TATGAGGTAGCAACTGCTATTACCT |
| | TTACAGTTAATGAGCAAGGTCAGGT |
| | TACTGTAAACGGCAAAGCAACTAAA |
| | GGTGACGCTCATATTGGCGTCGACC |
| | ACCACCACCACCACCACGGCGGCAG |
| | CGGCGGCAGCGGCGGTAGCGGCGGT |
| | AGCATGAAGATGGAAGAGCTGTTCA |
| | AGAAACACAAGATCGTTGCCGTGCT |
| | GCGTGCCAATAGTGTGGAAGAAGCG |
| | AAAAAGAAAGCGCTGGCGGTTTTCC |
| | TGGGCGGCGTTCATCTGATTGAAAT |
| | TACCTTTACCGTGCCGGATGCGGAT |
| | ACCGTGATTAAGGAACTGAGCTTTC |
| | TGAAGGAAATGGGCGCGATTATTGG |
| | TGCGGGCACCGTGACCAGCGTGGAG |
| | CAGTGCCGTAAAGCGGTGGAAAGTG |
| | GCGCCGAATTCATTGTGAGTCCGCA |
| | CCTGGACGAGGAAATTAGCCAATTT |
| | TGCAAGGAGAAGGGTGTGTTCTATA |
| | TGCCAGGCGTTATGACCCCGACCGA |
| | ACTGGTGAAAGCCATGAAACTGGGC |
| | CATACCATCTTAAAACTGTTTCCGG |
| | GTGAGGTGGTGGGTCCGCAGTTTGT |
| | TAAAGCGATGAAAGGTCCGTTTCCG |
| | AATGTGAAATTTGTGCCAACCGGCG |
| | GTGTTAATCTGGACAATGTGTGCGA |
| | ATGGTTCAAAGCGGGCGTGCTGGCC |
| | GTGGGCGTGGGCAGCGCGTTAGTGA |
| | AAGGCACCCCGGTGGAAGTGGCGGA |
| | AAAGGCCAAGGCGTTCGTTGAGAAG |
| | ATTCGTGGCTGCACCGAACATATGG |
| | GTGGCAGCGGAGGCTCTGGAGGTTC |
| | CGGCGGATCTGTGAGCAAGGGCGAG |
| | GCAGTGATCAAGGAGTTCATGCGGT |
| | TCAAGGTGCACATGGAGGGCTCCAT |
| | GAACGGCCACGAGTTCGAGATCGAG |
| | GGCGAGGGCGAGGGCCGCCCCTACG |
| | AGGGCACCCAGACCGCCAAGCTGAA |
| | GGTGACCAAGGGTGGCCCCCTGCCC |
| | TTCTCCTGGGACATCCTGTCCCCTC |
| | AGTTCATGTACGGCTCCAGGGCCTT |
| | CACCAAGCACCCCGCCGACATCCCC |
| | GACTACTATAAGCAGTCCTTCCCCG |
| | AGGGCTTCAAGTGGGAGCGCGTGAT |
| | GAACTTCGAGGACGGCGGCGCCGTG |
| | ACCGTGACCCAGGACACCTCCCTGG |
| | AGGACGGCACCCTGATCTACAAGGT |
| | GAAGCTTCGCGGCACCAACTTCCCT |
| | CCTGACGGCCCCGTAATGCAGAAGA |
| | AGACAATGGGCTGGGAAGCATCCAC |
| | CGAGCGGTTGTACCCCGAGGACGGC |
| | GTGCTGAAGGGCGACATTAAGATGG |
| | CCCTGCGCCTGAAGGACGGCGGTCG |
| | CTACCTGGCGGACTTCAAGACCACC |
| | TACAAGGCCAAGAAGCCCGTGCAGA |
| | TGCCCGGCGCCTACAACGTCGATCG |
| | CAAGTTGGACATCACCTCCCACAAC |
| | GAGGACTACACCGTGGTGGAACAGT |
| | ACGAACGCTCCGAGGGCCGCCACTC |
| | CACCGGCGGCATGGACGAGCTGTAC |
| | AAGTAA |
| Capture-Cage- | ATGGGCAGCAGCCATCATCATCATC |
| Green | ATCACGGCAGCGGCGATAGTGCTAC |
| SEQ ID NO: 22 | CCATATTAAATTCTCAAAACGTGAT |
| | GAGGACGGCAAAGAGTTAGCTGGTG |
| | CAACTATGGAGTTGCGTGATTCATC |
| | TGGTAAAACTATTAGTACATGGATT |
| | TCAGATGGACAAGTGAAAGATTTCT |
| | ACCTGTATCCAGGAAAATATACATT |
| | TGTCGAAACCGCAGCACCAGACGGT |
| | TATGAGGTAGCAACTGCTATTACCT |
| | TTACAGTTAATGAGCAAGGTCAGGT |
| | TACTGTAAACGGCAAAGCAACTAAA |
| | GGTGACGCTCATATTGGCGTCGACC |
| | ACCACCACCACCACCACGGCGGCAG |
| | CGGCGGCAGCGGCGGTAGCGGCGGT |
| | AGCATGAAGATGGAAGAGCTGTTCA |
| | AGAAACACAAGATCGTTGCCGTGCT |
| | GCGTGCCAATAGTGTGGAAGAAGCG |
| | AAAAAGAAAGCGCTGGCGGTTTTCC |
| | TGGGCGGCGTTCATCTGATTGAAAT |
| | TACCTTTACCGTGCCGGATGCGGAT |
| | ACCGTGATTAAGGAACTGAGCTTTC |
| | TGAAGGAAATGGGCGCGATTATTGG |
| | TGCGGGCACCGTGACCAGCGTGGAG |
| | CAGTGCCGTAAAGCGGTGGAAAGTG |
| | GCGCCGAATTCATTGTGAGTCCGCA |
| | CCTGGACGAGGAAATTAGCCAATTT |
| | TGCAAGGAGAAGGGTGTGTTCTATA |
| | TGCCAGGCGTTATGACCCCGACCGA |
| | ACTGGTGAAAGCCATGAAACTGGGC |
| | CATACCATCTTAAAACTGTTTCCGG |
| | GTGAGGTGGTGGGTCCGCAGTTTGT |
| | TAAAGCGATGAAAGGTCCGTTTCCG |
| | AATGTGAAATTTGTGCCAACCGGCG |
| | GTGTTAATCTGGACAATGTGTGCGA |
| | ATGGTTCAAAGCGGGCGTGCTGGCC |
| | GTGGGCGTGGGCAGCGCGTTAGTGA |
| | AAGGCACCCCGGTGGAAGTGGCGGA |
| | AAAGGCCAAGGCGTTCGTTGAGAAG |
| | ATTCGTGGCTGCACCGAACATATGG |
| | GTGGCAGCGGAGGCTCTGGAGGTTC |
| | CGGCGGATCTATGGTGTCGAAGGGG |
| | GAAGAGGATAACATGGCTAGTCTTC |
| | CAGCGACACACGAGCTTCACATTTT |
| | CGGTTCTATCAATGGAGTGGATTTC |
| | GACATGGTTGGCCAAGGAACAGGCA |
| | ACCCTAATGATGGATATGAAGAACT |
| | TAATCTTAAATCTACTAAAGGAGAC |
| | CTGCAATTCAGCCCCTGGATTCTGG |
| | TCCCTCACATTGGGTACGGTTTTCA |
| | CCAGTATCTTCCATATCCGGACGGT |
| | ATGTCTCCTTTCCAAGCGGCTATGG |
| | TGGACGGCTCGGGCTATCAAGTCCA |
| | TCGTACCATGCAGTTTGAAGATGGC |
| | GCGTCACTGACTGTGAATTACCGTT |
| | ACACATACGAGGGTAGTCATATCAA |
| | GGGAGAGGCCCAAGTCAAGGGAACG |
| | GGTTTTCCCGCCGATGGGCCAGTAA |
| | TGACAAATTCTCTTACCGCTGCCGA |
| | TTGGTGTCGTAGTAAAAAAACATAC |
| | CCAAACGATAAGACCATTATCTCAA |
| | CGTTCAAGTGGAGTTACACAACCGG |
| | GAACGGAAAGCGCTACCGTTCCACC |
| | GCACGCACGACTTACACGTTCGCGA |
| | AGCCAATGGCCGCTAATTACCTGAA |
| | AAATCAGCCTATGTACGTCTTCCGT |
| | AAGACTGAGTTAAAGCACAGTAAGA |
| | CAGAGCTGAACTTCAAGGAATGGCA |
| | GAAGGCGTTTACAGACGTAATGGGT |
| | ATGGATGAGTTGTATAAGTAG |
| BiotynCage-Red | ATGGGCCTAAATGATATCTTTGAAG |
| SEQ ID NO: 23 | CACAGAAAATCGAATGGCACGAAGG |
| | TGGGAGCGGGGGCTCGGGCGGAAGT |
| | CACCATCATCACCATCACGGCAGCG |
| | GCGATAGTGCTACCCATATTAAATT |
| | CTCAAAACGTGATGAGGACGGCAAA |
| | GAGTTAGCTGGTGCAACTATGGAGT |
| | TGCGTGATTCATCTGGTAAAACTAT |
| | TAGTACATGGATTTCAGATGGACAA |
| | GTGAAAGATTTCTACCTGTATCCAG |
| | GAAAATATACATTTGTCGAAACCGC |
| | AGCACCAGACGGTTATGAGGTAGCA |
| | ACTGCTATTACCTTTACAGTTAATG |
| | AGCAAGGTCAGGTTACTGTAAACGG |
| | CAAAGCAACTAAAGGTGACGCTCAT |
| | ATTGGCGTCGACGGTGGCAGCGGCG |
| | GGAGTGGAGGTTCTGGTGGGTCAAT |
| | GAAGATGGAAGAGCTGTTCAAGAAA |
| | CACAAGATCGTTGCCGTGCTGCGTG |
| | CCAATAGTGTGGAAGAAGCGAAAAA |
| | GAAAGCGCTGGCGGTTTTCCTGGGC |
| | GGCGTTCATCTGATTGAAATTACCT |
| | TTACCGTGCCGGATGCGGATACCGT |
| | GATTAAGGAACTGAGCTTTCTGAAG |
| | GAAATGGGCGCGATTATTGGTGCGG |
| | GCACCGTGACCAGCGTGGAGCAGTG |
| | CCGTAAAGCGGTGGAAAGTGGCGCC |
| | GAATTCATTGTGAGTCCGCACCTGG |
| | ACGAGGAAATTAGCCAATTTTGCAA |
| | GGAGAAGGGTGTGTTCTATATGCCA |
| | GGCGTTATGACCCCGACCGAACTGG |
| | TGAAAGCCATGAAACTGGGCCATAC |
| | CATCTTAAAACTGTTTCCGGGTGAG |
| | GTGGTGGGTCCGCAGTTTGTTAAAG |
| | CGATGAAAGGTCCGTTTCCGAATGT |
| | GAAATTTGTGCCAACCGGCGGTGTT |
| | AATCTGGACAATGTGTGCGAATGGT |
| | TCAAAGCGGGCGTGCTGGCCGTGGG |
| | CGTGGGCAGCGCGTTAGTGAAAGGC |
| | ACCCCGGTGGAAGTGGCGGAAAAGG |
| | CCAAGGCGTTCGTTGAGAAGATTCG |
| | TGGCTGCACCGAACATATGGGTGGC |
| | AGCGGAGGCTCTGGAGGTTCCGGCG |
| | GATCTGTGAGCAAGGGCGAGGCAGT |
| | GATCAAGGAGTTCATGCGGTTCAAG |
| | GTGCACATGGAGGGCTCCATGAACG |
| | GCCACGAGTTCGAGATCGAGGGCGA |
| | GGGCGAGGGCCGCCCCTACGAGGGC |
| | ACCCAGACCGCCAAGCTGAAGGTGA |
| | CCAAGGGTGGCCCCCTGCCCTTCTC |
| | CTGGGACATCCTGTCCCCTCAGTTC |
| | ATGTACGGCTCCAGGGCCTTCACCA |
| | AGCACCCCGCCGACATCCCCGACTA |
| | CTATAAGCAGTCCTTCCCCGAGGGC |
| | TTCAAGTGGGAGCGCGTGATGAACT |
| | TCGAGGACGGCGGCGCCGTGACCGT |
| | GACCCAGGACACCTCCCTGGAGGAC |
| | GGCACCCTGATCTACAAGGTGAAGC |
| | TTCGCGGCACCAACTTCCCTCCTGA |
| | CGGCCCCGTAATGCAGAAGAAGACA |
| | ATGGGCTGGGAAGCATCCACCGAGC |
| | GGTTGTACCCCGAGGACGGCGTGCT |
| | GAAGGGCGACATTAAGATGGCCCTG |
| | CGCCTGAAGGACGGCGGTCGCTACC |
| | TGGCGGACTTCAAGACCACCTACAA |
| | GGCCAAGAAGCCCGTGCAGATGCCC |
| | GGCGCCTACAACGTCGATCGCAAGT |
| | TGGACATCACCTCCCACAACGAGGA |
| | CTACACCGTGGTGGAACAGTACGAA |
| | CGCTCCGAGGGCCGCCACTCCACCG |
| | GCGGCATGGACGAGCTGTACAAGTA |
| | A |
| BiotynCage-Green | ATGGGCCTAAATGATATCTTTGAAG |
| SEQ ID NO: 24 | CACAGAAAATCGAATGGCACGAAGG |
| | TGGGAGCGGGGGCTCGGGCGGAAGT |
| | CACCATCATCACCATCACGGCAGCG |
| | GCGATAGTGCTACCCATATTAAATT |
| | CTCAAAACGTGATGAGGACGGCAAA |
| | GAGTTAGCTGGTGCAACTATGGAGT |
| | TGCGTGATTCATCTGGTAAAACTAT |
| | TAGTACATGGATTTCAGATGGACAA |
| | GTGAAAGATTTCTACCTGTATCCAG |
| | GAAAATATACATTTGTCGAAACCGC |
| | AGCACCAGACGGTTATGAGGTAGCA |
| | ACTGCTATTACCTTTACAGTTAATG |
| | AGCAAGGTCAGGTTACTGTAAACGG |
| | CAAAGCAACTAAAGGTGACGCTCAT |
| | ATTGGCGTCGACGGTGGCAGCGGCG |
| | GGAGTGGAGGTTCTGGTGGGTCAAT |
| | GAAGATGGAAGAGCTGTTCAAGAAA |
| | CACAAGATCGTTGCCGTGCTGCGTG |
| | CCAATAGTGTGGAAGAAGCGAAAAA |
| | GAAAGCGCTGGCGGTTTTCCTGGGC |
| | GGCGTTCATCTGATTGAAATTACCT |
| | TTACCGTGCCGGATGCGGATACCGT |
| | GATTAAGGAACTGAGCTTTCTGAAG |
| | GAAATGGGCGCGATTATTGGTGCGG |
| | GCACCGTGACCAGCGTGGAGCAGTG |
| | CCGTAAAGCGGTGGAAAGTGGCGCC |
| | GAATTCATTGTGAGTCCGCACCTGG |
| | ACGAGGAAATTAGCCAATTTTGCAA |
| | GGAGAAGGGTGTGTTCTATATGCCA |
| | GGCGTTATGACCCCGACCGAACTGG |
| | TGAAAGCCATGAAACTGGGCCATAC |
| | CATCTTAAAACTGTTTCCGGGTGAG |
| | GTGGTGGGTCCGCAGTTTGTTAAAG |
| | CGATGAAAGGTCCGTTTCCGAATGT |
| | GAAATTTGTGCCAACCGGCGGTGTT |
| | AATCTGGACAATGTGTGCGAATGGT |
| | TCAAAGCGGGCGTGCTGGCCGTGGG |
| | CGTGGGCAGCGCGTTAGTGAAAGGC |
| | ACCCCGGTGGAAGTGGCGGAAAAGG |
| | CCAAGGCGTTCGTTGAGAAGATTCG |
| | TGGCTGCACCGAACATATGGGTGGC |
| | AGCGGAGGCTCTGGAGGTTCCGGCG |
| | GATCTATGGTGTCGAAGGGGGAAGA |
| | GGATAACATGGCTAGTCTTCCAGCG |
| | ACACACGAGCTTCACATTTTCGGTT |
| | CTATCAATGGAGTGGATTTCGACAT |
| | GGTTGGCCAAGGAACAGGCAACCCT |
| | AATGATGGATATGAAGAACTTAATC |
| | TTAAATCTACTAAAGGAGACCTGCA |
| | ATTCAGCCCCTGGATTCTGGTCCCT |
| | CACATTGGGTACGGTTTTCACCAGT |
| | ATCTTCCATATCCGGACGGTATGTC |
| | TCCTTTCCAAGCGGCTATGGTGGAC |
| | GGCTCGGGCTATCAAGTCCATCGTA |
| | CCATGCAGTTTGAAGATGGCGCGTC |
| | ACTGACTGTGAATTACCGTTACACA |
| | TACGAGGGTAGTCATATCAAGGGAG |
| | AGGCCCAAGTCAAGGGAACGGGTTT |
| | TCCCGCCGATGGGCCAGTAATGACA |
| | AATTCTCTTACCGCTGCCGATTGGT |
| | GTCGTAGTAAAAAAACATACCCAAA |
| | CGATAAGACCATTATCTCAACGTTC |
| | AAGTGGAGTTACACAACCGGGAACG |
| | GAAAGCGCTACCGTTCCACCGCACG |
| | CACGACTTACACGTTCGCGAAGCCA |
| | ATGGCCGCTAATTACCTGAAAAATC |
| | AGCCTATGTACGTCTTCCGTAAGAC |
| | TGAGTTAAAGCACAGTAAGACAGAG |
| | CTGAACTTCAAGGAATGGCAGAAGG |
| | CGTTTACAGACGTAATGGGTATGGA |
| | TGAGTTGTATAAGTAG |
| BiotynCage-Blue | ATGGGCCTAAATGATATCTTTGAAG |
| SEQ ID NO: 25 | CACAGAAAATCGAATGGCACGAAGG |
| | TGGGAGCGGGGGCTCGGGCGGAAGT |
| | CACCATCATCACCATCACGGCAGCG |
| | GCGATAGTGCTACCCATATTAAATT |
| | CTCAAAACGTGATGAGGACGGCAAA |
| | GAGTTAGCTGGTGCAACTATGGAGT |
| | TGCGTGATTCATCTGGTAAAACTAT |
| | TAGTACATGGATTTCAGATGGACAA |
| | GTGAAAGATTTCTACCTGTATCCAG |
| | GAAAATATACATTTGTCGAAACCGC |
| | AGCACCAGACGGTTATGAGGTAGCA |
| | ACTGCTATTACCTTTACAGTTAATG |
| | AGCAAGGTCAGGTTACTGTAAACGG |
| | CAAAGCAACTAAAGGTGACGCTCAT |
| | ATTGGCGTCGACGGTGGCAGCGGCG |
| | GGAGTGGAGGTTCTGGTGGGTCAAT |
| | GAAGATGGAAGAGCTGTTCAAGAAA |
| | CACAAGATCGTTGCCGTGCTGCGTG |
| | CCAATAGTGTGGAAGAAGCGAAAAA |
| | GAAAGCGCTGGCGGTTTTCCTGGGC |
| | GGCGTTCATCTGATTGAAATTACCT |
| | TTACCGTGCCGGATGCGGATACCGT |
| | GATTAAGGAACTGAGCTTTCTGAAG |
| | GAAATGGGCGCGATTATTGGTGCGG |
| | GCACCGTGACCAGCGTGGAGCAGTG |
| | CCGTAAAGCGGTGGAAAGTGGCGCC |
| | GAATTCATTGTGAGTCCGCACCTGG |
| | ACGAGGAAATTAGCCAATTTTGCAA |
| | GGAGAAGGGTGTGTTCTATATGCCA |
| | GGCGTTATGACCCCGACCGAACTGG |
| | TGAAAGCCATGAAACTGGGCCATAC |
| | CATCTTAAAACTGTTTCCGGGTGAG |
| | GTGGTGGGTCCGCAGTTTGTTAAAG |
| | CGATGAAAGGTCCGTTTCCGAATGT |
| | GAAATTTGTGCCAACCGGCGGTGTT |
| | AATCTGGACAATGTGTGCGAATGGT |
| | TCAAAGCGGGCGTGCTGGCCGTGGG |
| | CGTGGGCAGCGCGTTAGTGAAAGGC |
| | ACCCCGGTGGAAGTGGCGGAAAAGG |
| | CCAAGGCGTTCGTTGAGAAGATTCG |
| | TGGCTGCACCGAACATATGGGTGGC |
| | AGCGGAGGCTCTGGAGGTTCCGGCG |
| | GATCTATGGTAAGCAAGGGAGAAGA |
| | ACTGTTTACAGGAGTTGTTCCTATC |
| | TTAGTTGAACTTGACGGCGACGTTA |
| | ACGGCCACAAGTTTTCCGTGAGCGG |
| | AGAGGGTGAGGGCGATGCCACTTAC |
| | GGTAAATTGACTTTAAAATTCATCT |
| | GCACTACCGGCAAACTTCCCGTTCC |
| | GTGGCCCACCTTGGTAACCACCCTT |
| | TCCTGGGGGGTCCAGTGCTTTGCAC |
| | GCTATCCAGATCACATGAAGCAACA |
| | CGATTTTTTTAAGAGTGCAATGCCG |
| | GAAGGTTATGTCCAAGAGCGCACTA |
| | TCTTTTTTAAGGATGACGGAAATTA |
| | CAAGACTCGCGCGGAAGTGAAGTTT |
| | GAGGGAGACACCCTTGTTAACCGCA |
| | TTGAATTGAAGGGCATCGACTTCAA |
| | GGAGGATGGAAACATCTTAGGGCAT |
| | AAACTTGAGTATAACTATTTTTCAG |
| | ATAATGTATATATCACAGCTGATAA |
| | ACAAAAGAATGGCATCAAAGCGAAT |
| | TTTAAAATCCGCCATAACATTGAGG |
| | ACGGAGGAGTGCAGTTAGCAGATCA |
| | TTACCAACAAAACACCCCGATTGGT |
| | GACGGCCCTGTACTTTTGCCAGACA |
| | ATCACTATTTGAGCACCCAAAGTAA |
| | ATTGTCGAAAGACCCTAACGAAAAG |
| | CGTGATCACATGGTCTTACTGGAAT |
| | TTGTCACAGCTGCGGGGATCACATT |
| | AGGTATGGATGAACTGTATAAGTAA |

It is appreciated based on the teachings provided herein and the skill of one in the art that modifications of any of the aforementioned sequences are similarly suitable. Illustratively, a monomer protein substructure is optionally 70% or more identical to any one of SEQ ID Nos: 11-25, optionally 80% or more identical to any one of SEQ ID Nos: 11-25, optionally 90% or more identical to any one of SEQ ID Nos: 11-25, optionally 95% or more identical to any one of SEQ ID Nos: 11-25, optionally 96% or more identical to any one of SEQ ID Nos: 11-25, optionally 97% or more identical to any one of SEQ ID Nos: 11-25, optionally 98% or more identical to any one of SEQ ID Nos: 11-25, optionally 99% or more identical to any one of SEQ ID Nos: 11-25.

### Target Protein

A multimeric protein structure that expresses a capture sequence is capable of binding, optionally specifically binding, a target protein, optionally an antigen or an antibody. As such, a target protein as used in the processes or compositions as provided herein is optionally an antigen such as an antigen or fragment thereof that includes one more epitopes. Optionally a target protein is an antibody, or a fragment thereof, optionally a heavy chain, light chain, 1-3 CDR sequences, or other. It is appreciated that a target protein may include one or more post-translational modifications such as glycosylation, phosphorylation, sulfonation, or others.

The target protein optionally is a modification of a wild-type sequence such that the target protein is non-naturally occurring. Such modifications include the addition, subtraction or substitution or one or more amino acids optionally for the purpose of including an endonuclease restriction site, a site to add or remove a post-translational modification, or a tag for purification or labeling purposes (e.g. 6xHis tag, GST tag, addition of a fluorophore, etc.), among other reasons known in the art for protein identification, labeling, localization, purification, etc.

A target protein optionally includes one or more capture tags that are complementary to a capture sequence on a multimeric protein structure. Complementary in this sense means that the capture tag will bind to, optionally specifically bind to, the capture sequence, optionally with high affinity. A target protein optionally includes 1 capture tag, optionally 2 or more capture tags. A capture tag is optionally a multimeric or repeating amino acid or nucleic acid sequence, a vitamin, or other suitable tag sequence. Illustrative examples of a capture tag on a target protein includes but are not limited to avidin, biotin, SEQ ID NO: 26 (AHIVMVDAYKPTK), or SEQ ID NO: 27 (KLGDIEFIKVNKG). It should be recognized that SEQ ID NO: 26 is a complementary capture tag to the capture sequence of SEQ ID NO: 7 in that the two sequences will self-associate to form a complex that is then auto-linked by a covalent bond between a lysine on one unit and an aspartic acid on the other unit to form an isopeptide bond. Similarly, the capture tag sequence SEQ ID NO: 27 is complementary to capture sequence SEQ ID NO: 9 where a complex is formed that results in the formation of a covalent linkage between the capture tag and the capture sequence. Similar and specific high affinity interactions are optionally observed between avidin and biotin where a substructure protein is labeled with either avidin or biotin, and the target protein is labeled with the complementary capture tag of either biotin or avidin.

A target protein optionally includes 1 capture tag, optionally 2 capture tags, optionally capture 3 tags. A tag is optionally localized to an N-terminal end, a C-terminal end, an intermediate position, or other. Optionally, a target protein is expressed with one or more capture tags within the peptide sequence and is exposed at the N-terminal end or C-terminal end by cleavage of a portion of the protein sequence by a protease.

As set forth in the examples herein, target proteins can include antigens or antigenic materials, such as viral, parasitic or bacterial antigens. As set forth in the methods and the examples herein, the employment of an antigen or antigenic peptide as a target protein can allow isolation and purification of B cells and/or T cells endogenously responsive to the presented antigen. For example, as set forth herein, to study the response to the murine malaria model P. yoelii, use of the PyMSP1(19) membrane bound protein fragment as a target protein allows for isolation of B cells responsive to that pathogen through the use of the multimeric protein structure. As a specific example, the amino acid sequence for PyMSP1(19) is as follows:

(SEQ ID NO: 28)
MTMSPILGYWKIKGLVQPTRLLLEYLEEKYEEHLY
ERDEGDKWRNKKFELGLEFPNLPYYIDGDVKLTQS
MAIIRYIADKHNMLGGCPKERAEISMLEGAVLDIR
YGVSRIAYSKDFETLKVDFLSKLPEMLKMFEDRLC
HKTYLNGDHVTHPDFMLYDALDVVLYMDPMCLDAF
PKLVCFKKRIEAIPQIDKYLKSSKYIAWPLQGWQA
TFGGGDHPPKSDLVPRGSSMGMHIASIALNNLNKS
GLVGEGESKKILAKMLNMDGMDLLGVDPKHVCVDT
RDIPKNAGCFRDDNGTEEWRCLLGYKKGEGNTCVE
NNNPTCDINNGGCDPTASCQNAESTENSKKIICTC
KEPTPNAYYEGVFCSSSSTSSGAHIVMVDAYKPTK
GLENLYFQGVEHHHHHH.

In some instances, the target protein utilized can be a control protein. Introduction of a control target protein may be desirable to better assess results obtained with other target protein structures. In some instances, the target protein may be a negative control protein, i.e. a protein that B cells and/or T cells will not recognize. For example, as discussed above, the PyMSP1(19) can be used as a target protein in a murine model of malaria. As a control, an additional protein such asPyUIS4 that is not expressed in the asexual blood stage of malaria infections, and thus is not recognized in infected models, can be employed as a negative “decoy” control. It can further be appreciated that use of a different fluorophore between a positive target protein and a negative control (or decoy) arrangement can allow for both to operate simultaneously. For example, as set forth below, the PyUIS4 control (decoy) protein was incorporated in a green fluorescent protein multimeric protein structure negative control to a PyMSP1(19) incorporated in a mScarlet red fluorescent protein multimeric protein structure. As a specific example, the amino acid sequence of the PyUIS4 when fused with SEQ ID NO: 26 and a histidine hexamer is

(SEQ ID NO: 30)
MTMSPILGYWKIKGLVQPTRLLLEYLEEKYEEHLY
ERDEGDKWRNKKFELGLEFPNLPYYIDGDVKLTQS
MAIIRYIADKHNMLGGCPKERAEISMLEGAVLDIR
YGVSRIAYSKDFETLKVDFLSKLPEMLKMFEDRLC
HKTYLNGDHVTHPDFMLYDALDVVLYMDPMCLDAF
PKLVCFKKRIEAIPQIDKYLKSSKYIAWPLQGWQA
TFGGGDHPPKSDLVPRGSSMGSSHHHHHHSSGLVP
RGSHMVREKFGIRKRIKNFDDVNTPQDISLISPVE
NPYQEYYPEDYQEQYPE1SSDQY1EQPQKHYTKRF
LEQYTNSVQNDHTYSYSPTEEKYNTYYMAPDTHDE
YEKLFTDDQKEEINDNIVYHDELSDLMGEGHKIYS
MNDKPFDPYIAHIVMVDAYKPTKVD.

Other specific target proteins are also described herein. It will be appreciated that peptides or protein fragments associated with generating an immune response in human populations are of significant interest, such as with immune responses to SARS-CoV-2, influenza H1N1 and P. falciparum. For example, to assess immune responses to SARS-CoV-2, the target protein can comprise an adapted spike protein of SARS-CoV-2 that includes the ectodomain and trimerization regions fused at the C-terminus with a histidine octamer, a linker and the capture tag (SEQ ID NO: 26) as set forth in the amino acid sequence:

(SEQ ID NO: 32)
MFVFLVLLPLVSSQCVNLTTRTQLPPAYTNSFTRG
VYYPDKVFRSSVLHSTQDLFLPFFSNVTWFHAIHV
SGTNGTKRFDNPVLPFNDGVYFASTEKSNIIRGWI
FGTTLDSKTQSLLIVNNATNVVIKVCEFQFCNDPF
LGVYYHKNNKSWMESEFRVYSSANNCTFEYVSQPF
LMDLEGKQGNFKNLREFVFKNIDGYFKIYSKHTPI
NLVRDLPQGFSALEPLVDLPIGINITRFQTLLALH
RSYLTPGDSSSGWTAGAAAYYVGYLQPRTFLLKYN
ENGTITDAVDCALDPLSETKCTLKSFTVEKGIYQT
SNFRVQPTESIVRFPNITNLCPFGEVFNATRFASV
YAWNRKRISNCVADYSVLYNSASFSTFKCYGVSPT
KLNDLCFTNVYADSFVIRGDEVRQIAPGQTGKIAD
YNYKLPDDFTGCVIAWNSNNLDSKVGGNYNYLYRL
FRKSNLKPFERDISTEIYQAGSTPCNGVEGFNCYF
PLQSYGFQPTNGVGYQPYRVVVLSFELLHAPATVC
GPKKSTNLVKNKCVNFNFNGLTGTGVLTESNKKFL
PFQQFGRDIADTTDAVRDPQTLEILDITPCSFGGV
SVITPGTNTSNQVAVLYQDVNCTEVPVAIHADQLT
PTWRVYSTGSNVFQTRAGCLIGAEHVNNSYECDIP
IGAGICASYQTQTNSPGSASSVASQSIIAYTMSLG
AENSVAYSNNSIAIPTNFTISVTTEILPVSMTKTS
VDCTMYICGDSTECSNLLLQYGSFCTQLNRALTGI
AVEQDKNTQEVFAQVKQIYKTPPIKDFGGFNFSQI
LPDPSKPSKRSFIEDLLFNKVTLADAGFIKQYGDC
LGDIAARDLICAQKFNGLTVLPPLLTDEMIAQYTS
ALLAGTITSGWTFGAGAALQIPFAMQMAYRFNGIG
VTQNVLYENQKLIANQFNSAIGKIQDSLSSTASAL
GKLQDVVNQNAQALNTLVKQLSSNFGAISSVLNDI
LSRLDPPEAEVQIDRLITGRLQSLQTYVTQQLIRA
AEIRASANLAATKMSECVLGQSKRVDFCGKGYHLM
SFPQSAPHGVVFLHVTYVPAQEKNFTTAPAICHDG
KAHFPREGVFVSNGTHWFVTQRNFYEPQIITTDNT
FVSGNCDVVIGIVNNTVYDPLQPELDSFKEELDKY
FKNHTSPDVDLGDISGINASVVNIQKEIDRLNEVA
KNLNESLIDLQELGKYEQGSGYIPEAPRDGQAYVR
KDGEWVLLSTFLGRSLEVLFQGPGHHHHHHHHGGG
SGGGGSGGAHIVMVDAYKPTK.

To examine influenza H1N1, the target protein can comprise a region of the HA protein thereof including the ectodomain and trimerization regions with a hexa histidine tag, a linker and capture tag (SEQ ID NO: 26) fused thereto at the C-terminus as set forth in the amino acid sequence:

(SEQ ID NO: 34)
MKAILVVLLYTFATANADTLCIGYHANNSTDTVDT
VLEKNVTVTHSVNLLEDKHNGKLCKLRGVAPLHLG
KCNIAGWILGNPECESLSTASSWSYIVETPSSDNG
TCYPGDFIDYEELREQLSSVSSFERFEIFPKTSSW
PNHESNKGVTAACPHAGAKSFYKNLIWLVKKGNSY
PKLSKSYINDKGKEVLVLWGIHHPPTSADQQSLYQ
NEDTYVFVGSSRYSKKFKPEIAIRPKVRDQEGRMN
YYWTLVEPGDKITFEATGNLVVPRYAFAMERNAGS
GIIISDTPVHDCNTTCQTPKGAINTSLPFQNIHPI
TIGKCPKYVKSTKLRLATGLRNIPSIQSRGLFGAI
AGFIEGGWTGMVDGWYGYHHQNEQGSGYAADLKST
QNAIDEITNKVNSVIEKMNTQFTAVGKEFNHLEKR
ENLNKKVDDGFLDIWTYNAELLVLLENERTLDYHD
SNVKNLYEKVRSQLKNNAKEIGNGCFEFYHKCDNT
CMESVKNGTYDYPKYSEEAKLNREEIDGVKLESTR
IYQGGGGGGSSSSSSSSSGYIPEAPRDGQAYVRKD
GEWVLLSTFLGGSHHHHHHGGSGGSGGSAHIVMVD
AYKPTKG

To examine the response to the parasite Plasmodium falciparum, the target protein can comprise a region of the MSP1(19) protein fused with a capture tag (e.g. SEQ ID NO: 26) and a hexa-histidine domain both at the C-terminus as set forth in the amino acid sequence:

(SEQ ID NO: 36)
MAMTMSPILGYWKIKGLVQPTRLLLEYLEEKYEEH
LYERDEGDKWRNKKFELGLEFPNLPYYIDGDVKLT
QSMAIIRYIADKHNMLGGCPKERAEISMLEGAVLD
IRYGVSRIAYSKDFETLKVDFLSKLPEMLKMFEDR
LCHKTYLNGDHVTHPDFMLYDALDVVLYMDPMCLD
AFPKLVCFKKRIEAIPQIDKYLKSSKYIAWPLQGW
QATFGGGDHPPKSDLVPRGSSVGMNISQHQCVKKQ
CPENSGCFRHLDEREECKCLLNYKQEGDKCVENPN
PTCNENNGGCDADATCTEEDSGSSRKKITCECTKP
DSYPLFDGIFCSSSNTSSGAHIVMVDAYKPTKGLE
NLYFQGLEHHHHHH.

It should be also understood that in some instances it may be desired to include the capture tag in the monomer protein structure and the capture sequence in the target protein. In similar or different instances, it may be desired to include a complementary affinity sequence in the target protein instead or as well as in the monomer protein structure. Such rearrangements and similar are all within the scope of the complexes described herein.

Target proteins, similar to substructure proteins, are optionally produced by recombinant DNA expression efforts as recognized in the art. As such, a target protein sequence optionally includes one or more of an extra amino acid or multiple amino acids resulting from the insertion of a restriction endonuclease cleave site in the DNA, one or more protease cleavage sites, and one or more purification tags. A target protein may be coexpressed with associated purification tags, modifications, other proteins such as in a fusion peptide, or other modifications or combinations as recognized in the art. Illustrative purification tags include 6xHis, FLAG, biotin, ubiquitin, SUMO, or other tag known in the art. A purification tag is illustratively cleavable such as by linking to a target protein via an enzyme cleavage sequence that is cleavable by an enzyme known in the art illustratively including Factor Xa, thrombin, SUMOstar protein, TEV protease, or trypsin. It is further appreciated that chemical cleavage is similarly operable with an appropriate cleavable linker.

A monomer protein substructure, target protein, or any portion thereof, optionally further including a purification tag, linker, capture sequence, protease cleavage site, or other, are optionally formed by recombinant DNA expression methods. The identification of codon sequences in DNA/RNA from a known protein sequence are readily achieved by persons of ordinary skill in the art. Protein expression is illustratively accomplished from transcription of desired nucleic acid sequence, translation of RNA transcribed from desired nucleic acid sequence, modifications thereof, or fragments thereof. Protein expression is optionally performed in a cell-based system such as in E. coli, HeLa cells, or Chinese hamster ovary cells. Bacterial cells such as E. coli are commonly used, but if post-translational modifications are desired on one or more amino acids of a target protein, protein substructure or both, they may be expressed in a mammalian cell. It is appreciated that cell-free expression systems are similarly operable.

It is recognized that numerous variants, analogues, or homologues are within the scope of the present protein including amino acid substitutions, alterations, modifications, or other amino acid changes that increase, decrease, or do not alter the function of the substructure protein sequence or target protein sequence. Several post-translational modifications are similarly envisioned as within the scope of the present disclosure illustratively including incorporation of a non-naturally occurring amino acid, phosphorylation, glycosylation, addition of pendent groups such as biotinylation, fluorophores, lumiphores, radioactive groups, antigens, or other molecules.

Methods of recombinantly expressing a protein substructure or target protein nucleic acid or protein sequence or fragments thereof are also provided herein wherein a cell is transformed, transfected, or transduced with a desired nucleic acid sequence and cultured under suitable conditions that permit expression of the protein substructure or target protein nucleic acid sequence or protein either within the cell or secreted from the cell. Cell culture conditions are particular to cell type and expression vector. Culture conditions for particular vectors and cell types are within the level of skill in the art to design and implement without undue experimentation.

Recombinant or non-recombinant proteinase peptides or recombinant or non-recombinant proteinase inhibitor peptides or other non-peptide proteinase inhibitors can also be used in the expression of a substructure protein or target protein. Proteinase inhibitors are optionally modified to resist degradation, for example degradation by digestive enzymes and conditions. Techniques for the expression and purification of recombinant proteins are known in the art (see Sambrook Eds., Molecular Cloning: A Laboratory Manual 3^{rd} ed. (Cold Spring Harbor, N.Y. 2001).

Some aspects of the present disclosure are compositions containing monomer protein substructure (e.g., I3-01 monomer protein substructure (SEQ ID NO: 1)) or target protein nucleic acid that can be expressed as encoded polypeptides or proteins. The engineering of DNA segment(s) for expression in a prokaryotic or eukaryotic system may be performed by techniques generally known to those of skill in recombinant expression. It is believed that virtually any expression system may be employed in the expression of the claimed nucleic and amino sequences.

Generally speaking, it may be more convenient to employ as the recombinant polynucleotide a cDNA version of the polynucleotide. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the possibility of employing a genomic version of a particular gene (e.g. target protein) where desired is not excluded.

As used herein, the terms “engineered” and “recombinant” cells are synonymous with “host” cells and are intended to refer to a cell into which an exogenous DNA segment or gene, such as a cDNA or gene has been introduced. Therefore, engineered cells are distinguishable from naturally occurring cells that do not contain a recombinantly introduced exogenous DNA segment or gene. A host cell is optionally a naturally occurring cell that is transformed, transfected, or transduced with an exogenous DNA segment or gene or a cell that is not modified. A host cell preferably does not possess a naturally occurring gene encoding or similar to a target protein or protein substructure. Engineered cells are thus cells having a gene or genes introduced through the hand of man. Recombinant cells include those having an introduced cDNA or genomic DNA, and also include genes positioned adjacent to a promoter not naturally associated with the particular introduced gene.

To express a recombinant encoded polypeptide in accordance with the present disclosure one would prepare an expression vector that comprises a polynucleotide under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli .chi. 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda may be utilized in making a recombinant phage vector that can be used to transform host cells, such as E. coli LE392.

Further useful vectors include pIN vectors and pGEX vectors, for use in generating glutathione S-transferase (GST) soluble fusion proteins for later purification and separation or cleavage. Other suitable fusion proteins are those with β-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to microorganisms, cultures of cells derived from multicellular organisms may also be used as hosts. In principle, any such cell culture is operable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autographica californica nuclear polyhedrosis virus (AcNPV) is used as a vector to express foreign genes. The virus grows in Spodoptera frugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non- occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome-binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment that also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly(A) addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin. It is appreciated that numerous other selection systems are known in the art that are similarly operable in the present invention.

It is contemplated that the isolated nucleic acids of the disclosure may be “overexpressed”, i.e., expressed in increased levels relative to its natural expression in cells of its indigenous organism, or even relative to the expression of other proteins in the recombinant host cell. Such overexpression may be assessed by a variety of methods, including radio-labeling and/or protein purification. However, simple and direct methods are preferred, for example, those involving SDS-PAGE and protein staining or immunoblotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

Further aspects of the present disclosure concern the purification, and in particular embodiments, the substantial purification, of an encoded protein or peptide. The term “purified” or “isolated” protein or peptide as used herein, is intended to refer to a composition, isolatable from other components, wherein the protein or peptide is purified to any degree relative to its naturally-obtainable state, i.e., in this case, relative to its purity within a cell. A purified protein or peptide therefore also refers to a protein or peptide, free from the environment in which it may naturally occur.

Generally, “purified” or “isolated” will refer to a protein or peptide composition that has been subjected to fractionation to remove various other components, and which composition substantially retains its expressed biological activity. Where the term “substantially” purified is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure as based on knowledge in the art. These include, for example, determining the specific activity of an active fraction, or assessing the number of polypeptides within a fraction by SDS-PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS-PAGE (Capaldi et al., Biochem. Biophys. Res. Comm., 76:425, 1977). It will therefore be appreciated that under differing electrophoresis conditions, the apparent molecular weights of purified or partially purified expression products may vary.

Methods of obtaining a target protein or protein substructure illustratively include isolation of target protein or protein substructure from a host cell or host cell medium. Methods of protein isolation illustratively include column chromatography, affinity chromatography, gel electrophoresis, filtration, or other methods known in the art. Optionally, target protein or protein substructure is expressed with a tag operable for affinity purification. As described above, optionally, a purification tag is a 6x His tag. A 6x His tagged protein is illustratively purified by Ni-NTA column chromatography or using an anti-6x His tag antibody fused to a solid support (Geneway Biogech, San Diego, Calif.). Other tags and purification systems are similarly operable.

It is appreciated that a target protein or protein substructure is optionally not tagged. Purification is optionally achieved by methods known in the art illustratively including ion-exchange chromatography, affinity chromatography using anti-target protein or substructure protein antibodies, precipitation with salt such as ammonium sulfate, streptomycin sulfate, or protamine sulfate, reverse phase chromatography, size exclusion chromatography such as gel exclusion chromatography, HPLC, immobilized metal chelate chromatography, or other methods known in the art. One of skill in the art may select the most appropriate isolation and purification techniques without departing from the scope of this invention.

A target protein, protein substructure, or fragment thereof is optionally chemically synthesized. Methods of chemical synthesis have produced proteins greater than 600 amino acids in length with or without the inclusion of modifications such as glycosylation and phosphorylation. Methods of chemical protein and peptide synthesis illustratively include solid phase protein chemical synthesis. Illustrative methods of chemical protein synthesis are reviewed by Miranda, L P, Peptide Science, 2000, 55:217-26 and Kochendoerfer G G, Curr Opin Drug Discov Devel. 2001; 4(2):205-14, the contents of which are incorporated herein by reference.

As discussed above, one or more monomer protein substructures includes a capture sequence. Optionally, all protein substructures include a capture sequence. As such, many aspects a multimeric protein structure includes a plurality of capture sequence domains available for association with a target protein via the capture tag. The number of monomer protein substructures that include a capture sequence or the number of bound target proteins to a multimeric protein structure relative to the total number of such sites available is a target protein saturation level. A saturation level is optionally 1% or greater, optionally 1.6% or greater, optionally 5% or greater, optionally 10% or greater, optionally 20% or greater, optionally 30% or greater, optionally 40% or greater, optionally 50% or greater, optionally 60% or greater, optionally 70% or greater, optionally 80% or greater, optionally 90% or greater, optionally 99% or greater, optionally 100%.

A target protein, monomer protein substructure or both are optionally provided in a solvent, optionally water, optionally buffered water. A solvent optionally includes one or more salts. A salt is optionally present at a level of 1 mM to 500 mM, or greater, or any value or range there between. Optionally the level of salt is 1 mM or greater, optionally 10 mM or greater, optionally 50 mM or greater, optionally 100 mM or greater, optionally 200 mM or greater, optionally 300 mM or greater, optionally 400 mM or greater, optionally 500 mM or greater. Optionally, the level of salt is 200 mM to 500 mM, optionally 300 mM to 500 mM.

Processes of isolating, characterizing, identifying, or otherwise one or more immune cells as provided herein may include the decoration of a pre-purified protein multimeric protein structure with the target protein (e.g., antibody, antigen, etc.) that bears a capture tag (e.g., SPYTAG, SNOOPTAG, AVITAG, respectively) or in the case of the use of monomeric streptavidin as the capture sequence, with any target protein that is biotinylated, optionally uniformly biotinylated. Uncaptured molecules-of-interest are simply dialyzed away.

These monomeric protein substructures or self-assembled multimeric protein structures can easily be used alone or as part of a kit for identification, isolation, characterization or other desired use of an immune cell. These allow for orthologous capture systems that use covalent or high affinity non-covalent bonds. This can also allow for the capture of proteins with commonly used epitope tags by use of an adapter molecule with the monomeric streptavidin capture domain (which binds to biotin).

### Methods of Use

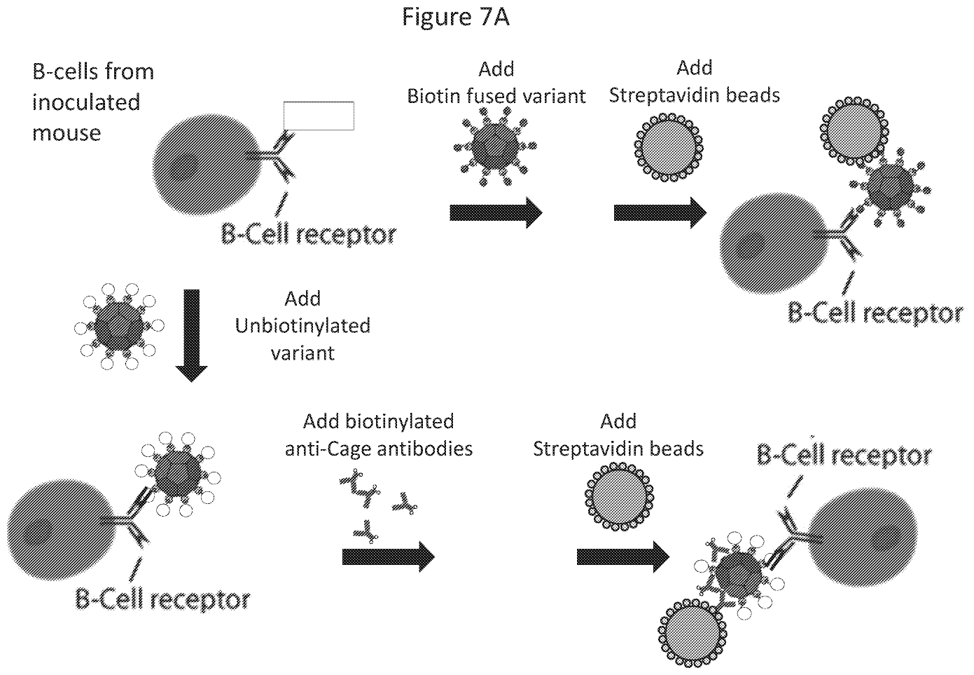

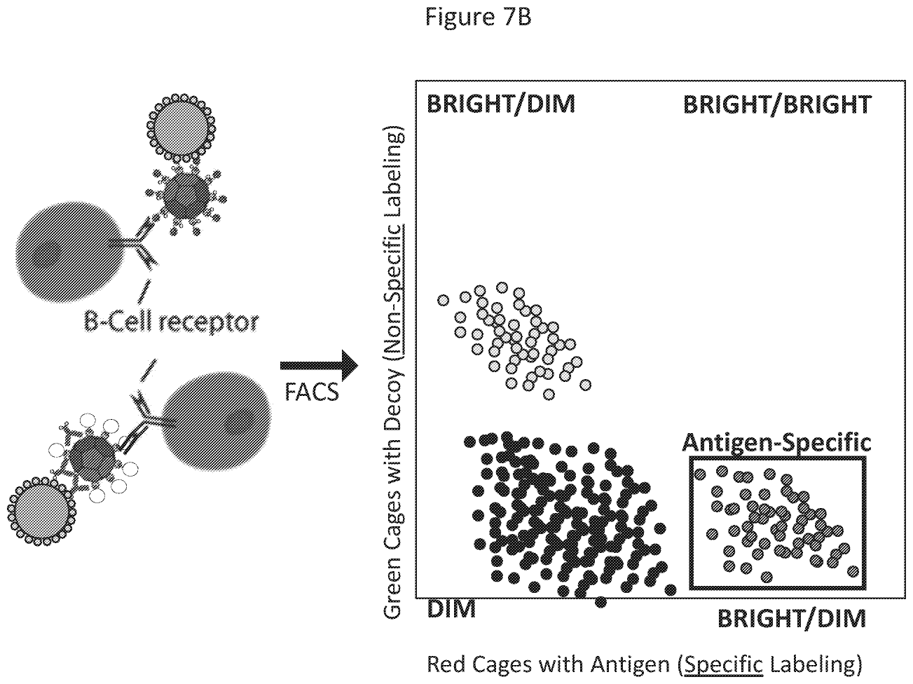

The multimeric protein structures can be used in methods to identify adaptive immune cells, such as B cells or T cells, that are responsive to a target protein antigen of choice. FIGS. 7A and 7B show an overview of possible applications of the multimeric protein structure and its resulting complex to isolate B cells. The methods include providing a multimeric protein structure as described herein with a capture sequence with a target protein antigen affixed with a corresponding a capture tag sequence. The two capture domains interact and thereby form a complex.

A population of cells can be incubated with the complex. In some instances, the population of cells includes adaptive immune cells. In other instances, the population of cells can be derived from a sample from a subject, such as a blood sample or tissue sample. As a specific example, the tissue may be a spleen or a lymph node. Adaptive immune cells responsive to the target protein or that recognize the target protein endogenously will recognize the tagged recombinant target protein present within the complex and freely associate therewith.

The complex can then be isolated, such as by chromatographic or cytometric means to provide separation. In some instances, isolation may include both means. For example, as described herein, antibodies or a further complementary affinity sequence tag can be utilized to link the complex to a solid support. In some examples, antibodies responsive to the multimeric protein structure (or monomer protein substructure) of the complex can be incubated therewith. The antibodies may be tagged, such as with a biotin tag, and then incubated with a binding partner to that tag, such as streptavidin or avidin in the case of biotin, wherein the binding partner is affixed to a solid support. Examples of the solid support include beads, such as magnetic beads, sepharose beads, glass beads, and agarose beads. In the case of utilizing magnetic beads, application of a magnetic field can be utilized for isolation of the complex and associated cells therewith.

In some aspects, the complex includes a complementary affinity sequence fused with the monomer protein substructure domains and the capture sequence domains. As described, the complementary affinity sequence responds to and binds a complementary binding partner. In some instances, the complementary affinity sequence is a biotin tag and the complementary binding partner is avidin or a derivative thereof. The complementary binding partner may be covalently coupled to a solid support.

Once the complex is incubated with cells and allowed to interact and couple to the solid support, isolation of associated immune cells can be performed. The methods as provided herein are capable of detecting, isolating, characterizing, identifying, or other desired outcome of one or more immune cells from a sample. An immune cell as used herein is optionally an adaptive immune cell. Optionally the adaptive immune cells are T cells and in certain other embodiments the adaptive immune cells are B cells.

Optionally, a B-cell is contacted with one or more complexes containing an antigen of interest (e.g. the target protein within the complex). The resulting complex-bound B-cell is optionally detected by one or more known techniques such as fluorescence-activated cell sorting (FACS) analysis. FACS analyses are illustratively described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

As is provided herein B-cells expressing B-cell receptors (BCR) that bind to a specific antigen/epitope can be non-destructively labeled and selected. This may optionally be accomplished by FACS by using a fluorescent cage (multimeric protein structure) (as provided herein) decorated with that antigen specific for the desired B-cell receptor (target protein), magnetic-activated cell sorting (MACS) when the cage is biotinylated or labeled with a specific, biotinylated antibody that allows binding of a streptavidin-coated magnetic bead as an example, or combinations thereof In instances where the expressed multimeric protein structure comprises a complementary affinity sequence, such as a biotin sequence, the expressed multimeric protein structure may be incubated with a complementary binding partner affixed to a solid support, such as streptavidin or avidin affixed to a bead, optionally a magnetic bead. The similar approaches enable the non-destructive labeling and selection of T-cells through the use of a recombinant major histocompatibility complex class I (MHC-I) complex loaded with a specific peptide antigen. For example, MHC heavy chain with fused capture sequence and beta-2 microglobulin are refolded in the presence of a peptide epitope of interest. This tripartite complex is incubated with the capture scaffold and used to label T-cells that are specific for that peptide. For both applications, the provided multimeric protein structure reagents can have fluorescence intensities that are 10-times brighter than existing tetramers, and will allow for all commonly used downstream applications of isolated B- and T-cells.

As such provided are methods for detecting the presence or absence of an immune cell. The immune cell is optionally a B-cell or T-cell. Optionally, an immune cell is a B-cell that expresses a BCR specific for an antigen-of-interest, optionally a protein or a portion of a protein expressed by an infectious agent, optionally a protein selectively related to a disease state causatively or otherwise. A process optionally includes contacting a sample with a cage as provided herein wherein the cage is linked to an antigen or other target protein of interest. Optionally, the cage includes a fluorophore within or bound to the cage structure that enables fluorescent detection of the presence or absence of a desired cell or cell type.

The methods of isolating B and T cells as described herein can be further adapted to serve related purposes. For example, in some instances, the basic steps of the described method can be utilized to confirm or deny whether a subject has immunity to a target protein, or more generally a virus, pathogen or bacterium represented by the target protein, or has been previously exposed to such. By isolating B cells or T cells specific to a target protein antigen, it can be determined whether the subject from which the cells are derived has a natural immunity to that particular antigen. For example, cells derived from a subject that are responsive to the target protein in the complex without any known or deliberate pre-exposure to the antigen provides a positive data point in determining whether the subject is already immune to the antigen, potentially by prior unknown exposure.

The methods of isolating B cells and T cells as described herein further provide methods and opportunities to develop B cell and T cell cultures, each being primed against the target antigen. Once isolated either by isolation of a streptavidin- or avidin-tagged bead or by flow cytometry or both, the isolated cells can be established in an in vitro culture. Also, cells isolated with the multimeric protein structure can endocytose and degrade the complex over time. As such, these cells can be cultured and expanded with irradiated fibroblast feeder cells. Expanded cells can be cloned out by limited dilution and the antibodies produced by those cells can be assessed (see, e.g., Carbonetti et al., J. Immunol. Methods 448: 66-73 (2017)). Cells can also be provided free antigen in excess to compete for binding. In some instances, B cells can be plated in small dishes pre-seeded with stromal cells with an input cell density of ˜100 B cells/cm². and cultured in a suitable medium [e.g. RPMI 1640 with 5% serum, 55 μM 2-mercaptoethanol, 2 mM L-glutamine, 100 U/ml penicillin, 100 μg/ml streptomycin, 10 mM HEPES, 1 mM sodium pyruvate and 1% MEM nonessential amino acids], supplemented with recombinant cytokines such as IL-2, IL-4, IL-21, and BAFF for ˜8 days. During this period, cells are fed by aspirating half of the old medium and replacing the same volume with fresh medium with cytokines. More detailed protocols for establishing such are found at e.g. Su et al., J. Immunol. 197: 4163-4176 (2016) and/or Carbonetti et al., J. Immunol. Methods 448: 66-73 (2017).

Alternatively, RNA can be isolated from cells and used to generate recombinant monoclonal antibodies. In cases where isolated cells will be subjected to single cell RNA sequencing (scRNA-seq), there is no anticipated consequence to the presence of this complex, as one cell is lysed in an independent well of a 96-well plate and the variable sequences of the heavy and light chain IgG are targeted for sequencing. These sequences are then cloned into an expression vector for the production of recombinant monoclonal antibodies (see, e.g., Rizzetto et al., Bioinformatics 34: 2846-2847 (2018)) . Briefly, mRNA from is collected and used as a template to generate a cDNA with the use of reverse transcriptase, followed by PCR with primers to VH, VL and/or VK domains and subsequent fusion into an IgG vector to produce a monoclonal chimera. Antigen binding is utilized to identify specific library members. Further details are found at e.g., Guthmiller et al., Methods Mol. Biol. 1904: 109-145 (2019) and Lei et al. Front. Microbiol. 10:672 (2019).

It some instances, the methods described herein can be modified to assess antigenicity of a protein fragment or a peptide. For example, test peptides can be utilized as target proteins within the complex. Cells from a subject already immune or exposed to the full protein from which the peptide is derived can then be incubated with target test peptides and the affinity of B cell or T cells that results allows for determination of which peptides generate better adaptive immune cell binding.

The methods described herein can utilize one or more target protein-multimeric structure protein complexes. Through the use of different fluorophores, multiple complexes can be incubated with a collection of cells. For example, as described above, a control protein can be utilized in a complex with one fluorophore, such as a red fluorophore, and an investigatory protein can be utilized with a second fluorophore, such as a green fluorophore. Therefore, multiple complexes can be included in the methods described herein and utilization of the corresponding fluorophores can provide an approach to assess each complex independently and in concert.

A sample is optionally any sample that does or may contain an immune cell. Optionally a sample is a tissue, such as tissue obtained from the spleen, lymph node or other organ of a subject. Optionally, a tissue is blood, serum, plasma, cancer tissue, marrow, skin, or any other tissue as is found in an organism, optionally a human. Optionally, a sample is a secretion from a tissue such as from a mucus membrane. A sample may be obtained from a subject by any desired means. Optionally, blood can be collected by venipuncture. Plasma may be collected from blood by centrifugation or other desired means. A tissue sample may be obtained by biopsy, swab or other collection.

As used herein, a “subject” is defined as an organism (such as a human, non-human primate, equine, bovine, murine, or other mammal), or a cell.

An infectious agent is optionally a virus, bacterial, parasite, or other organism. An infectious agent is optionally a virus optionally a virus that is or causes one or more viral diseases that include, but are not limited to: HIV, AIDS, AIDS Related Complex, chickenpox (Varicella), common cold, cytomegalovirus, Colorado tick fever, dengue fever, Ebola, hand, foot and mouth disease, hepatitis, herpes simplex, herpes zoster, HPV (human papillomavirus), influenza (Flu), Lassa fever, measles, Marburg hemorrhagic fever, infectious mononucleosis, mumps, norovirus, poliomyelitis, progressive multifocal leukoencephalopathy, rabies, rubella, SARS, Mers, SARS-CoV-2, smallpox (Variola), viral encephalitis, viral gastroenteritis, viral meningitis, viral pneumonia, West Nile disease, and yellow fever. Optionally, an infectious agent is one that is or causes HIV/AIDS and viral infections that may cause cancer. The main viruses associated with human cancers are human papillomavirus, hepatitis B and hepatitis C virus, Epstein-Barr virus, and human T-lymphotropic virus.

Examples of bacterial infectious agent include or cause, but are not limited to: anthrax, bacterial meningitis, botulism, Brucellosis, campylobacteriosis, cat scratch disease, cholera, diphtheria, epidemic typhus, gonorrhea, impetigo, legionellosis, leprosy (Hansen's Disease), leptospirosis, listeriosis, Lyme disease, melioidosis, rheumatic fever, MRSA, nocardiosis, pertussis, plague, pneumococcal pneumonia, psittacosis, Q fever, rocky mountain spotted fever (RMSF), salmonellosis, scarlet fever, shigellosis, Syphilis, tetanus, trachoma, tuberculosis, tularemia, typhoid fever, typhus, and urinary tract infections.

Optionally an infectious agent is a parasite that causes one or more parasitic infections. Illustrative examples include, but not limited to a parasite that causes: African trypanosomiasis, amebiasis, ascariasis, bab esiosis, Chagas disease, clonorchiasis, cryptosporidiosis, cysticercosis, diphyllobothriasis, dracunculiasis, Echinococcosis, enterobiasis, fascioliasis, fasciolopsiasis, filariasis, free-living amebic infection, giardiasis, gnathostomiasis, hymenolepiasis, isosporiasis, kala-azar, leishmaniasis, malaria, metagonimiasis, myiasis, onchocerciasis, pediculosis, pinworm infection, scabies, schistosomiasis, taeniasis, toxocariasis, toxoplasmosis, trichinellosis, trichinosis, trichuriasis, trichomoniasis, and trypanosomiasis; fungal infectious diseases such as but not limited to: aspergillosis, blastomycosis, candidiasis, coccidioidomycosis, cryptococcosis, histoplasmosis, tinea pedis; prion infectious diseases such as but not limited to: transmissible spongiform encephalopathy, bovine spongiform encephalopathy, Creutzfeldt-Jakob disease, Kuru-Fatal Familial Insomnia, and Alpers syndrome.

A protein related to a disease state causatively or otherwise, is optionally a protein related to an autoimmune disease or condition. Illustrative examples of an autoimmune disease or condition include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG₄-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

The processes as provided herein are optionally non-destructed to the target cell of interest enabling further use by subsequent techniques as may be desired. Illustrative examples of downstream applications of B-cell labeling and capture can include the sequencing of the heavy chain and light chain coding sequences for the production of recombinant antibodies, the fusion of selected B-cells with cancer cell lines to produce hybridomas, etc.

Various aspects of the present invention are illustrated by the following non-limiting examples. The examples are for illustrative purposes and are not a limitation on any practice of the present invention. It will be understood that variations and modifications can be made without departing from the spirit and scope of the invention. Reagents illustrated herein are commercially available, and a person of ordinary skill in the art readily understands where such reagents may be obtained.

### EXAMPLES

### Example 1: Production of Protein Substructures and Multimers Thereof

The current accepted model to isolate B cells involves biotinylation of a recombinant antigen of interest and the subsequent formation of a tetramer with streptavidin-phycoerythrin (PE) by careful control of the molar amounts of each. (see, e.g., Rahe et al., Viral Immunol. 31: 1-10 (2018)). Such an approach provides varying results, possibly due to hindrance of the antigen by the proximity and amount of biotin and streptavidin. A design for better antigen presentation was thus developed.

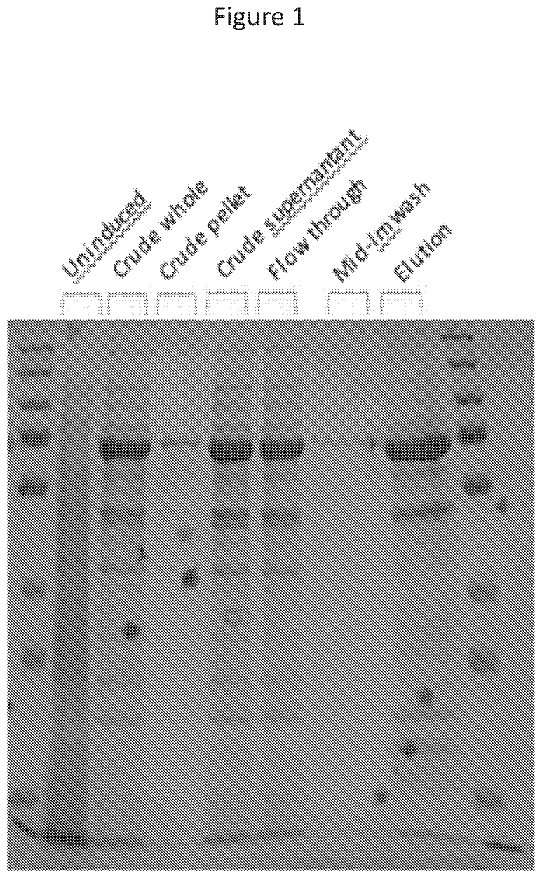

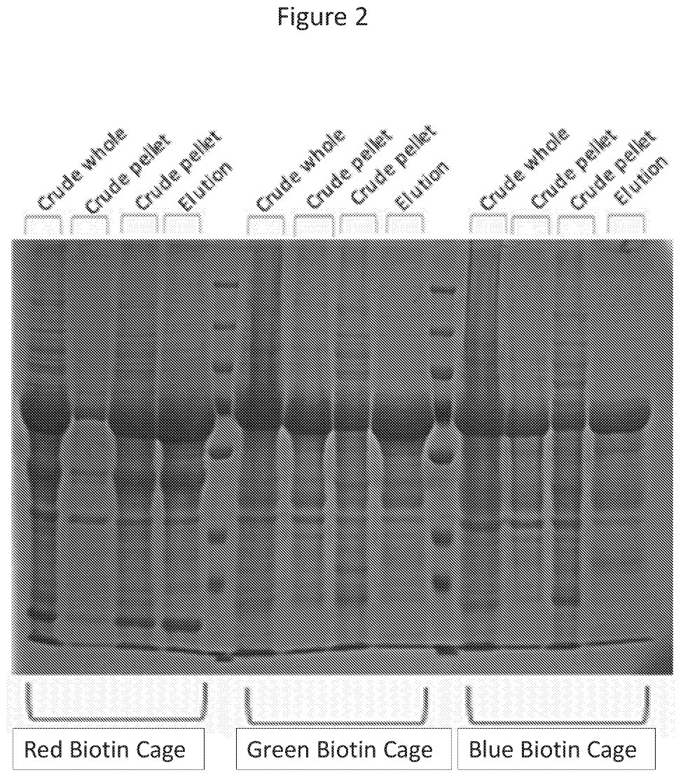

Polynucleotide sequences of SEQ ID Nos: 21-25 that respectively expresses fluorescent monomer protein substructures of SEQ ID Nos: 16-20 were each ligated into a modified pET28b+ expression vector. The recombinant protein was expressed in CodonPlus(DE3) strain of E. coli grown in 1-3 L of LB broth in shaker flasks. To produce the soluble protein, the culture was grown to an OD₆₀₀ of 0.6 and protein expression was induced by addition of 0.5 mM IPTG (final concentration) and incubated at 37° C. for 3 hours. The cells were then harvested and suspended in 10 mL of Low Imidazole Buffer (25 mM Tris-Cl pH7.5@ RT, 500 mM NaCl, 10 mM imidazole, 1 mM DTT, 1 mM benzamidine, and 10% v/v glycerol) and lysed by 3 rounds of sonication with each round consisting of 30 pulses at 30% amplitude and 50% duty cycle (Model 450 Branson Digital Sonifier, Disruptor Horn). The crude extract was spun at 3234 xg for 20 minutes at 4° C. The supernatant was incubated with 0.5 ml of Ni-NTA resin (Thermo Scientific, Cat# 88223), which was equilibrated in Low Imidazole Buffer on a nutator for 1 hour at 4 C. The resin was washed with 20 CV Mid Imidazole Buffer (25 mM Tris-Cl pH 7.5@ RT, 500 mM NaCl, 50 mM imidazole, 1 mM DTT, 1 mM benzamidine, and 10% glycerol) then eluted with 2 CV of High Imidazole Buffer (25 mM Tris-Cl pH 7.5@ RT, 500 mM NaCl, 300 mM imidazole, 1 mM DTT, 1 mM benzamidine, and 10% v/v glycerol). The resulting fractions were then run on a 12% SDS-PAGE with results are shown in FIG. 1.

The coding sequence for the Red Biotin Cage (mScarlet), Green Biotin Cage (mNeonGreen), and Blue Biotin Cage (mTurquoise2) (biotinylated monomer protein substructure variants) were synthesized (Twist Biosciences) then cloned into a modified pET28 vector. The constructs were transformed into E. coli BL21 (DE3) CodonPlus and were grown in 1 L of LB media at 37 C. To induce expression of biotinylated Cages, IPTG was added to the culture to a final concentration of 0.5 mM and allowed to grow at 23° C. for 16 hours. The cells were then harvested and suspended in 30 mL of Low-Imidazole Buffer (25 mM Tris-Cl pH 7.5@ RT, 500 mM NaCl, 10 mM imidazole, 1 mM DTT, 1 mM benzamidine, and 10% v/v glycerol) and lysed by 3 rounds of sonication with each round consisting of 30 pulses at 60% amplitude and 50% duty cycle (Model 450 Branson Digital Sonifier, Disruptor Horn). The crude extract was spun at 15000×g for 20 minutes at 4° C. The supernatant was incubated with 3 ml of Ni-NTA resin (Thermo Scientific, Cat# 88223), which was equilibrated in Low Imidazole Buffer on a nutator for 1 hour at 4° C. The resin was washed with 10 CV Mid Imidazole Buffer (25 mM Tris-Cl pH 7.5@ RT, 500 mM NaCl, 50 mM imidazole, 1 mM DTT, 1 mM benzamidine, and 10% v/v glycerol) then eluted with 3 CV of High Imidazole Buffer (25 mM Tris-Cl pH 7.5@ RT, 500 mM NaCl, 300 mM imidazole, 1 mM DTT, 1 mM benzamidine, and 10% v/v glycerol). The resulting fractions were then run on a 12% SDS-PAGE.

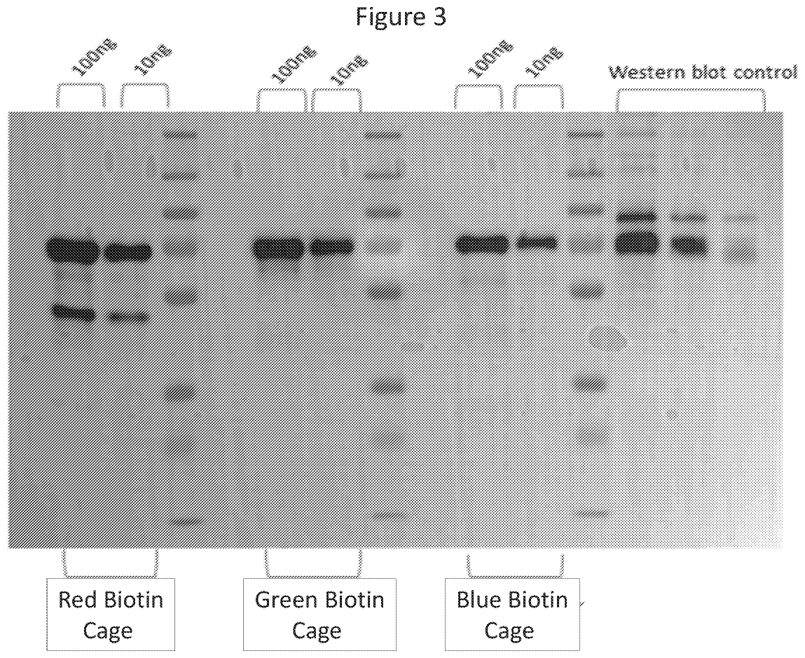

To assess if the biotinylated monomer protein substructures were in fact biotinylated upon expression in E. coli, the purified monomer protein substructures were run on a 10% SDS-PAGE, transferred to blotting paper, then probed using streptavidin-HRP (results shown in FIG. 3). All three colors of monomer protein substructures were found to be biotinylated.

The individual monomer protein substructures self-assembled into a plurality of multimeric protein structures. To further purify the multimeric protein structures, anion exchange chromatography was performed using a 20 mL bed volume of Q-Sepharose resin that was equilibrated in T100 pH 8.5 Solution (Buffer A). The column was then washed using 3 CV Buffer A, and multimeric protein structures were eluted using a linear gradient from 0-100% Buffer B (20 mM Tris-Cl pH 8.5@ RT, 1000 mM NaCl, 1 mM DTT, and 10% v/v glycerol) over 20 CV. The elution pool was exhaustively dialyzed into 20 mM Tris pH 8.0@ RT, 100 mM NaCl, 1 mM DTT, and 10% glycerol. Lastly, the purified multimeric protein structures was concentrated to 2-5 mg/ml using Amicon Ultra Centrifugal Filters (Fisher Scientific Cat# UFC9-003-08).

### Example 2: Target Protein Expression—B-Cell Antigens

A portion of Plasmodium yoelii Merozoite Surface Protein 1 (PyMSP1), which is commonly known as the 19 kD fragment (PyMSP1(19)) was recombinantly expressed. This target protein also contains common purification epitope tags, as well as Capture-Tag (SEQ ID NO: 26) to enable its covalently attachment to unbiotinylated and the biotinylated variants. PyMSP1(19)::Capture-Tag readily binds and forms a covalent bond with Capture-Cage (unbiotinylated) at room temperature in 1-2 hours. PyMSP1(19) is a well-established B-cell antigen for P. yoelii blood stage infections, and serves as a positive control. The amino acid sequence for PyMSP1(19) as used in this example is as follows:

(SEQ ID NO: 28)
MTMSPILGYWKIKGLVQPTRLLLEYLEEKYEEHLY
ERDEGDKWRNKKFELGLEFPNLPYYIDGDVKLTQS
MAIIRYIADKHNMLGGCPKERAEISMLEGAVLDIR
YGVSRIAYSKDFETLKVDFLSKLPEMLKMFEDRLC
HKTYLNGDHVTHPDFMLYDALDVVLYMDPMCLDAF
PKLVCFKKRIEAIPQIDKYLKSSKYIAWPLQGWQA
TFGGGDHPPKSDLVPRGSSMGMHIASIALNNLNKS
GLVGEGESKKILAKMLNMDGMDLLGVDPKHVCVDT
RDIPKNAGCFRDDNGTEEWRCLLGYKKGEGNTCVE
NNNPTCDINNGGCDPTASCQNAESTENSKKIICTC
KEPTPNAYYEGVFCSSSSTSSGAHIVMVDAYKPTK
GLENLYFQGVEHHHHHH.

The DNA sequence encoding the above is as follows:

(SEQ ID NO: 29)
ATGACCATGTCCCCTATACTAGGTTATTGGAAAAT
TAAGGGCCTTGTGCAACCCACTCGACTTCTTTTGG
AATATCTTGAAGAAAAATATGAAGAGCATTTGTAT
GAGCGCGATGAAGGTGATAAATGGCGAAACAAAAA
GTTTGAATTGGGTTTGGAGTTTCCCAATCTTCCTT
ATTATATTGATGGTGATGTTAAATTAACACAGTCT
ATGGCCATCATACGTTATATAGCTGACAAGCACAA
CATGTTGGGTGGTTGTCCAAAAGAGCGTGCAGAGA
TTTCAATGCTTGAAGGAGCGGTTTTGGATATTAGA
TACGGTGTTTCGAGAATTGCATATAGTAAAGACTT
TGAAACTCTCAAAGTTGATTTTCTTAGCAAGCTAC
CTGAAATGCTGAAAATGTTCGAAGATCGTTTATGT
CATAAAACATATTTAAATGGTGATCATGTAACCCA
TCCTGACTTCATGTTGTATGACGCTCTTGATGTTG
TTTTATACATGGACCCAATGTGCCTGGATGCGTTC
CCAAAATTAGTTTGTTTTAAAAAACGTATTGAAGC
TATCCCACAAATTGATAAGTACTTGAAATCCAGCA
AGTATATAGCATGGCCTTTGCAGGGCTGGCAAGCC
ACGTTTGGTGGTGGCGACCATCCTCCAAAATCGGA
TCTGGTTCCGCGTGGATCTTCCATGGGGATGCATA
TTGCGTCAATTGCATTGAATAACTTAAACAAATCT
GGCTTAGTCGGAGAAGGGGAGTCGAAAAAAATTTT
GGCAAAAATGTTAAACATGGATGGAATGGATTTAC
TTGGCGTCGATCCAAAGCACGTTTGCGTTGATACG
CGCGATATTCCTAAAAATGCAGGCTGTTTTCGTGA
CGATAATGGTACCGAAGAATGGCGTTGTCTTCTTG
GATACAAGAAAGGTGAAGGGAATACCTGCGTAGAG
AACAATAATCCCACTTGCGATATCAATAACGGCGG
GTGTGACCCAACCGCCTCTTGCCAAAACGCCGAGT
CAACGGAGAACTCTAAGAAGATCATTTGCACCTGC
AAAGAACCGACACCAAATGCCTATTATGAGGGGGT
CTTCTGTTCTTCGTCATCCACTAGTTCAGGCGCCC
ACATCGTGATGGTGGACGCCTACAAGCCGACGAAG
GGTCTCGAGAACCTGTACTTCCAGGGAGTCGAGCA
CCACCACCACCACCACTGA.

We also recombinantly expressed and purified the non-membrane bound portion of Plasmodium yoelii Upregulated in Infectious Sporozoites 4 (PyUIS4) also with common purification tags and Capture-Tag (capture tag SEQ ID NO: 26). This control target protein similarly binds and forms a covalent bond with the capture sequence in the multimeric protein structures (Capture-Cage) in identical conditions. PyUIS4 is not produced in blood stage infections of P. yoelii (only in the sporozoite and liver stages), and thus serves as a negative control to identify cells that bind non-specifically with biotinylated/unbiotinylated variants. The amino acid sequence of the PyUIS4 used in this example is:

(SEQ ID NO: 30)
MTMSPILGYWKDCGLVQPTRLLLEYLEEKYEEHLY
ERDEGDKWRNKKFELGLEFPNLPYYIDGDVKLTQS
MAIIRYIADKHNMLGGCPKERAEISMLEGAVLDIR
YGVSRIAYSKDFETLKVDFLSKLPEMLKMFEDRLC
HKTYLNGDHVTHPDFMLYDALDVVLYMDPMCLDAF
PKLVCFKKRIEAIPQIDKYLKSSKYIAWPLQGWQA
TFGGGDHPPKSDLVPRGSSMGSSHHHHHHSSGLVP
RGSHMVREKFGIRKRIKNFDDVNTPQDISLISPVE
NPYQEYYPEDYQEQYPEISSDQYIEQPQKHYTKRF
LEQYTNSVQNDHTYSYSPTEEKYNTYYMAPDTHDE
YEKLFTDDQKEEINDNIVYHDELSDLMGEGHKIYS
MNDKPFDPYIAHIVMVDAYKPTKVD.

The DNA sequence encoding the above is as follows:

(SEQ ID NO: 31)
ATGACCATGTCCCCTATACTAGGTTATTGGAAAAT
TAAGGGCCTTGTGCAACCCACTCGACTTCTTTTGG
AATATCTTGAAGAAAAATATGAAGAGCATTTGTAT
GAGCGCGATGAAGGTGATAAATGGCGAAACAAAAA
GTTTGAATTGGGTTTGGAGTTTCCCAATCTTCCTT
ATTATATTGATGGTGATGTTAAATTAACACAGTCT
ATGGCCATCATACGTTATATAGCTGACAAGCACAA
CATGTTGGGTGGTTGTCCAAAAGAGCGTGCAGAGA
TTTCAATGCTTGAAGGAGCGGTTTTGGATATTAGA
TACGGTGTTTCGAGAATTGCATATAGTAAAGACTT
TGAAACTCTCAAAGTTGATTTTCTTAGCAAGCTAC
CTGAAATGCTGAAAATGTTCGAAGATCGTTTATGT
CATAAAACATATTTAAATGGTGATCATGTAACCCA
TCCTGACTTCATGTTGTATGACGCTCTTGATGTTG
TTTTATACATGGACCCAATGTGCCTGGATGCGTTC
CCAAAATTAGTTTGTTTTAAAAAACGTATTGAAGC
TATCCCACAAATTGATAAGTACTTGAAATCCAGCA
AGTATATAGCATGGCCTTTGCAGGGCTGGCAAGCC
ACGTTTGGTGGTGGCGACCATCCTCCAAAATCGGA
TCTGGTTCCGCGTGGATCTTCCATGGGCAGCAGCC
ATCATCATCATCATCACAGCAGCGGCCTGGTGCCG
CGCGGCAGCCATATGGTGCGTGAAAAATTTGGTAT
TCGCAAACGTATTAAAAATTTCGATGACGTGAACA
CCCCGCAGGACATTAGCCTGATTAGCCCGGTGGAG
AATCCGTACCAGGAATATTACCCGGAGGACTACCA
GGAGCAGTATCCGGAGATTAGCAGCGACCAGTACA
TCGAACAGCCGCAGAAGCATTACACCAAACGCTTC
CTGGAGCAGTATACCAACAGCGTGCAGAACGATCA
CACCTATAGCTACAGCCCGACCGAGGAGAAGTACA
ACACCTACTACATGGCCCCGGATACCCACGACGAG
TACGAGAAACTGTTCACCGATGACCAGAAAGAAGA
AATTAATGATAATATTGTGTATCATGATGAACTGA
GTGACCTGATGGGCGAGGGCCATAAAATCTACAGC
ATGAATGATAAACCGTTTGATCCGTACATTGCACA
CATCGTTATGGTAGATGCATATAAACCAACTAAAG
TCGACTAA.

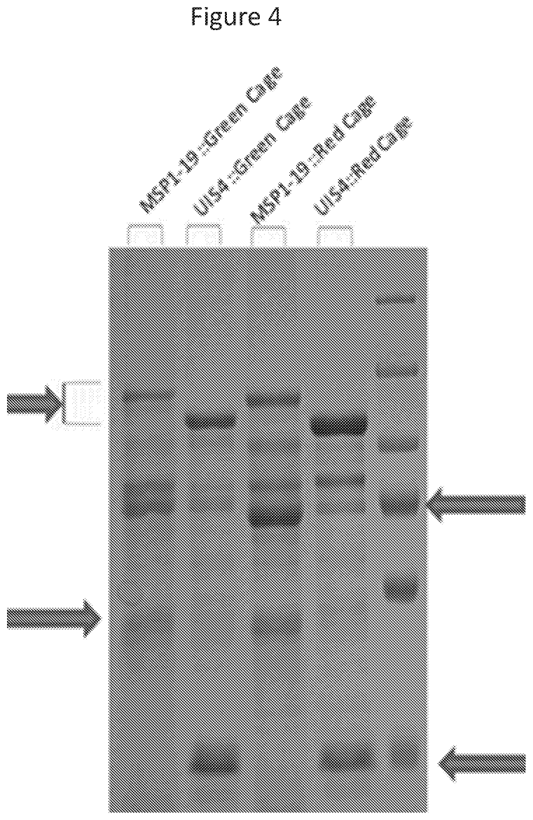

Antigens (UIS4 and MSP1-19) fused with a Capture-Tag were bound to Capture-Cage-Green (green fluorescent protein variant SEQ ID NO: 17) or Capture-Cage-Red (red fluorescent protein variant SEQ ID NO: 16) by incubation at room temperature at a molar ratio of 1.2 to 1 (antigen to Capture-Cage monomer protein substructure). To assess the level of saturation, samples were loaded on to a 10% SDS-PAGE which is shown in FIG. 4. MSP1-19 bound cages are 40-50% saturated and UIS4 bound cages are 90% saturated (see, lanes 3 and 4 of FIG. 4).

### Example 3: Production of Biotin-Labeled Anti-Capture-Cage IgG Antibodies

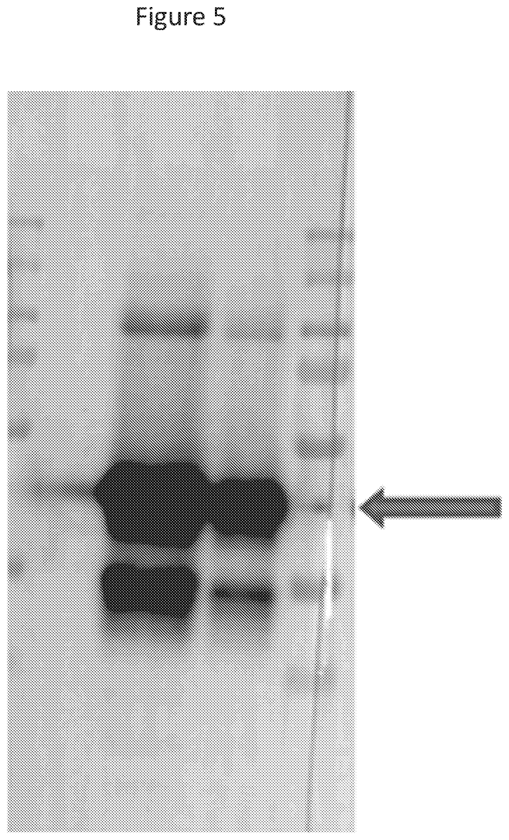

A polyclonal antibody was made in rabbits against purified recombinant Capture-Cage (SEQ ID NO: 11) by Pocono Rabbit Farm and Laboratory (Canadensis, Pa.). A total of 0.5 mg of Capture-Cage was injected per rabbit over an 84 day (Fusion Protein) protocol. Antibodies were purified from antisera using standard ammonium sulfate cuts. Next, the IgG was purified further using anion exchange chromatography using a 20 mL bed volume of Q-Sepharose resin that was equilibrated in Buffer A (20 mM Tris-Cl pH8.0@ RT). The column was then washed using 3 column volumes (CV) Buffer A then eluted using a linear gradient from 0-100% B (Buffer B: 20 mM Tris-Cl pH 8.0@ RT, 1000 mM NaCl) over 20 CV. The elution fractions containing the antibody were pooled and exhaustively dialyzed into 1×PBS. Lastly, the purified protein was concentrated to 3.9 mg/ml using Amicon Ultra Centrifugal Filters (Fisher Scientific Cat# UFC9-003-08). To verify that the IgG recognizes Capture-Cage, recombinant Capture-Cage was run on a 10% SDS-PAGE gel, transferred to blotting paper, then probed (western blotting) using the purified IgG as the primary antibody and goat anti-rabbit IgG-HRP as the secondary antibody. Results are illustrated in FIG. 5.

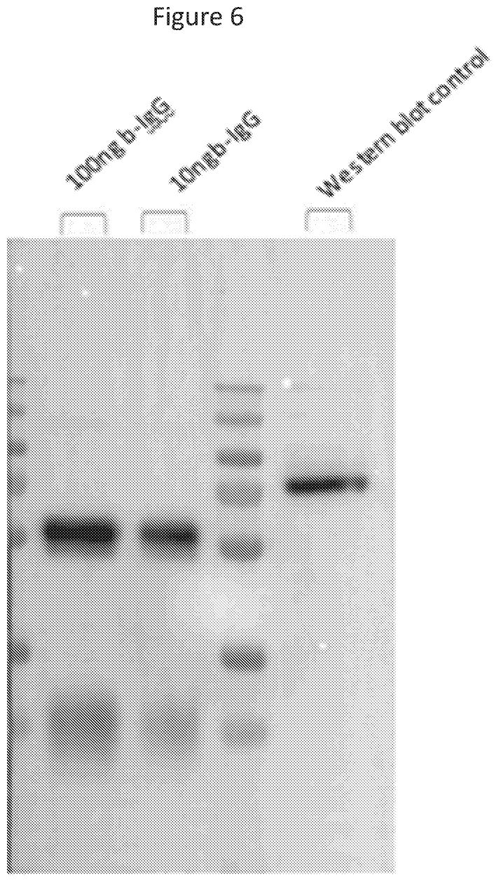

The purified IgG fraction was labeled with biotin using the EZ-Link Sulfo-NHS-Biotin crosslinker (Fisher Scientific, cat#:PI21217) at a molar ratio of 10 to 1 (crosslinker to IgG) at room temperature for 2 hours. Excess linker was removed by dialysis into 1×PBS. To verified that biotin labeling had occurred, the labeled IgG was run on a 10% SDS-PAGE, transferred to blotting paper, then probed using streptavidin-HRP (results shown in FIG. 6). Both heavy chain and light chain were found to be biotinylated.

### Example 4: B-Cell Labeling and Capture

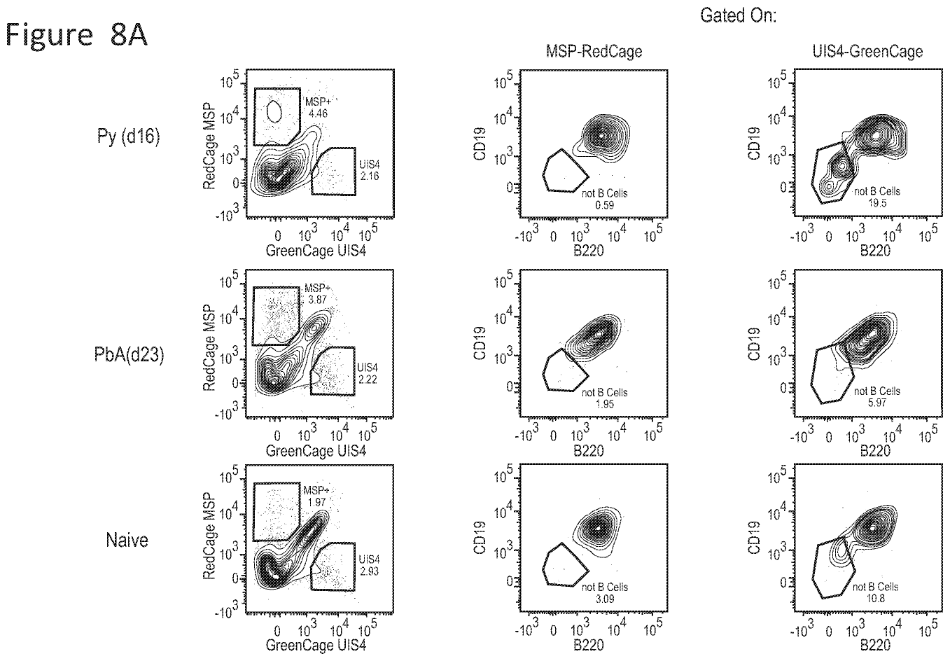

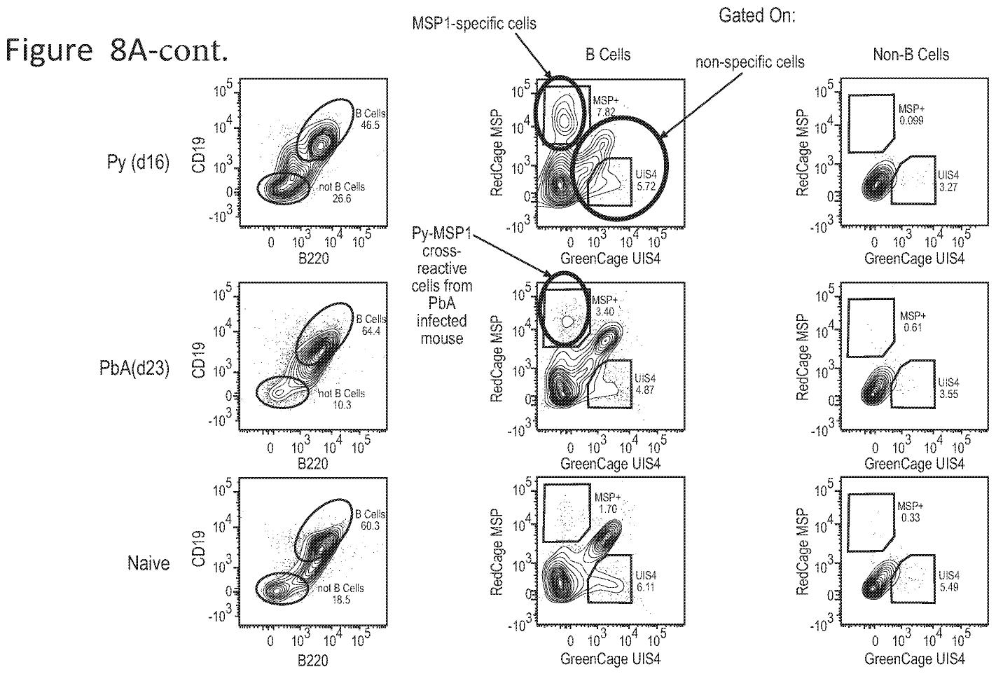

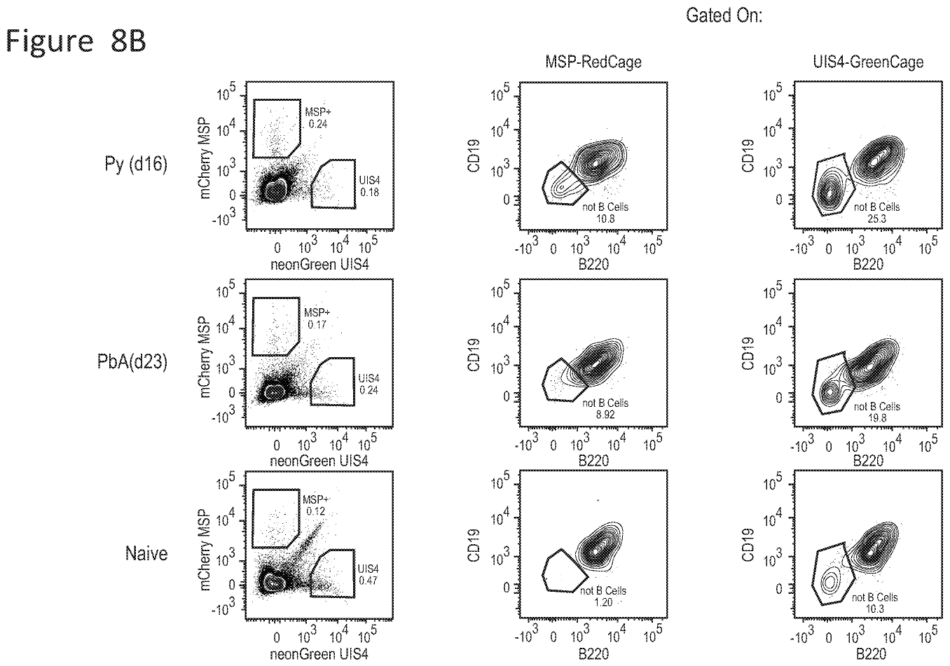

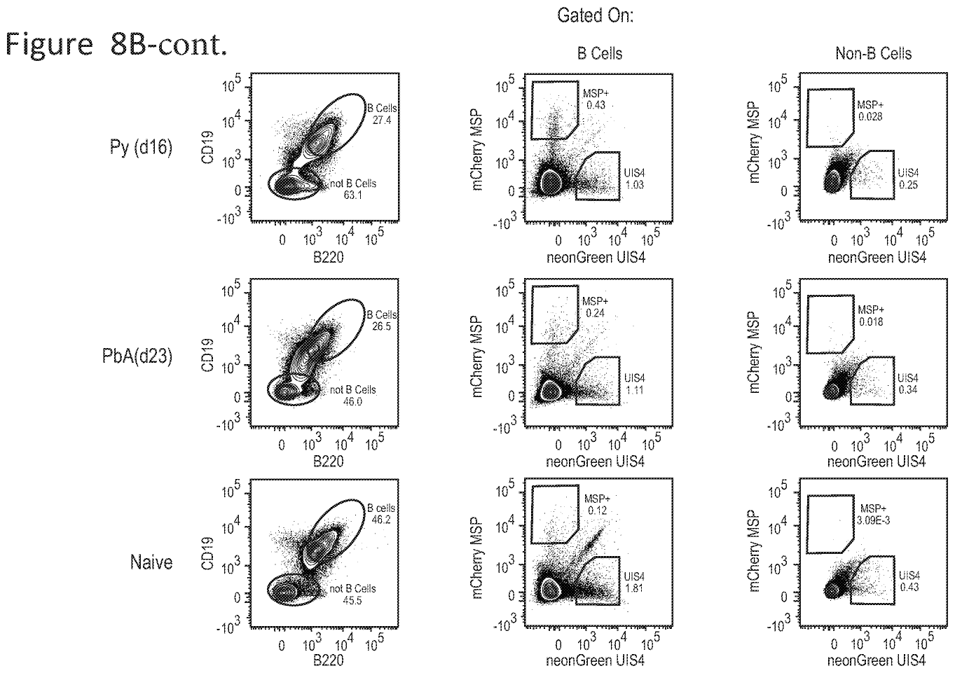

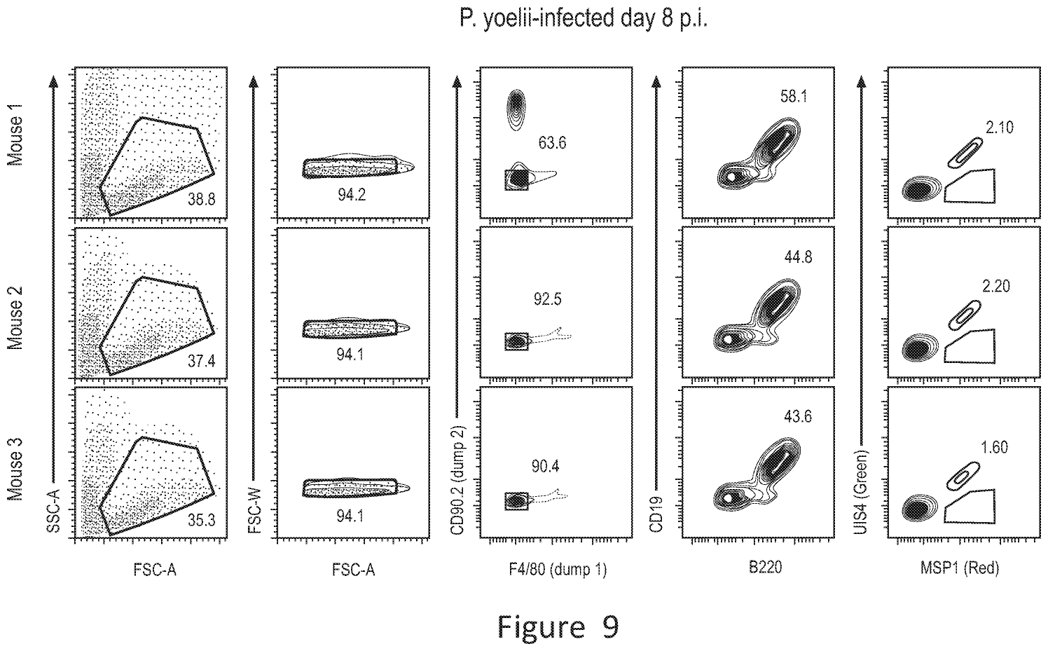

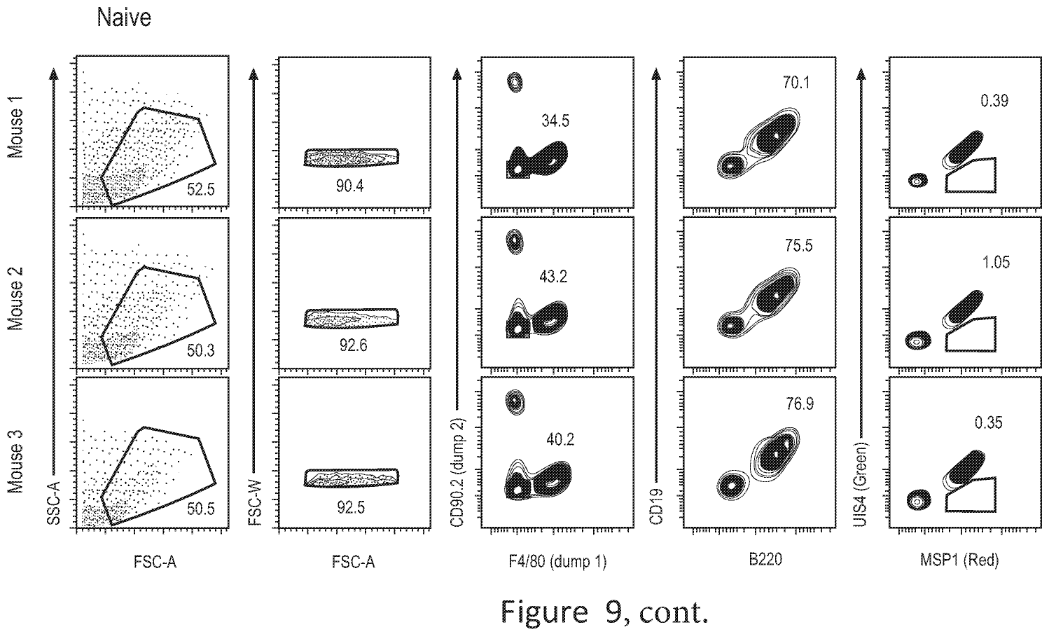

An overview of the strategy to B cell isolation is presented in FIGS. 7A and 7B. Mice were infected with Plasmodium yoelii or the related pathogen Plasmodium berghei, or were left uninfected (naïve). Cell suspensions derived from the spleen of these mice were stained with decoy Capture-Cage: :PyUIS4 for 10 minutes at room temperature. Then Capture-Cage: :MSP1(19) was added to allow for specific binding while on ice for 30 minutes. Cell suspensions were washed and then stained with a biotinylated anti-aldolase antibody for 30 minutes at 4 C. Cells were then washed and labeled with streptavidin-conjugated magnetic beads for 20 minutes. Cells with a bound magnetic bead were then selected by the possel function on AutoMACS (Miltenyi Biotec). Antibodies to known B-cell antigens (B220, CD19) were added and allowed to bind for 20 minutes at 4 C, and cells were subjected to FACS. B-cells derived from a mouse infected with P. yoelii were readily detected with Capture-Cage::MSP1(19) (7.82% of cells), as were those derived from mice infected with P. berghei (3.40%), which surpassed the number of B-cells from naïve mice (1.70%). Comparable numbers of cells bound the decoy Capture-Cage::UIS4 in all sample types (P. yoelii-infected mice, P. berghei-infected mice, naïve mice). Data are illustrated in FIGS. 8 and 9. FIG. 8A shows a comparison between the antigen present in a biotin-streptavidin tetramer model and with the multimeric protein structure system discussed herein. As seen on the right of FIG. 8A, the complex provided for significantly better isolation of B cells than the tetramer model. FIG. 8B shows the results when the run-through was examined, confirming that the complex retained the B cells through the washes. FIG. 9 shows the repeated isolation of specific B cells in three P.yoelii inoculated mice using the unbiotinylated variant (Ab biotinylation).

### Example 5: T-Cell Labeling and Capture

Unbiotinylated (Capture-Cage) and biotinylated (BiotynCage) variants as provided above and otherwise herein can be loaded with refolded MHC Class I complexes for non-destructive T-cell labeling and capture. These two variants are expected to be ˜10 times brighter than those described by Krishnamurty, et al., Immunity, 2016, Aug. 16;45(2):402-14 or those avaliable from the NIH Tetramer Core Facility based at Emory University (http://tetramer.yerkes.emory.edu) the best tetramer currently available, and will position up to five MHC-I complexes on the same face of the cage to potentially improve binding avidity. This methodology can extend to include other MHC-I heavy chain allele types, MHC-II complexes or to link other immune reagents.

### Example 6. Capture-Cage Staining and Flow Cytometry

A single cell suspension (2×10⁷ cells) of splenocytes was incubated with 1.25 ug decoy (control target protein) (Capture-cage::UIS4 mScarlet) in FACs buffer (PBS+2% FCS+2 mM EDTA) for 10 minutes at room temperature. The cells were then incubated with 1.25 μg Capture-Cage::MSP1-mNeoGreen in FACs buffer on ice for 30 minutes (no wash between). The cells were then washed twice with FACs buffer and centrifuged at 1600 rpm for 8 minutes. The cells were then incubated with 1 μg biotinylated anti-cage antibody for 30 minutes on ice, followed by one wash with FACs buffer.

Cells were then labelled with 20 μL streptavidin- microbeads (from miltenyi Biotec) and incubated for 15 minutes in a refrigerator. The cells were next washed with 2 mL of FACs buffer and centrifuged at 1800 rpm for 10 minutes. The supernatant was then aspirated and the cells resuspended in 500 μL MACs buffer.

The cells then proceeded to magnetic separation. First, a MACs LS column was placed in a magnetic field and washed with 3 mL of MACs buffer. The cell suspension was then applied onto the column. Unlabeled cells that pass through were collected in a new tube. The column was then washed with 3 mL of MACs buffer and the column was removed from the magnetic field and placed on a new collection tube. 3 mL of MACs buffer was added onto the column and the magnetically labeled cells then flushed out by firmly pushing a plunger into the column. The labeled cells (MSP1-postive cells) were then washed twice with buffer and the number of cells were counted.

Cells were then stained with Zombie NIR dye (1:1000 dilution in PBS) at room temperature for 20 minutes. The cells were then washed once with FACs buffer and stained with CD19 and B220 in FACs buffer on ice for 30 minutes. Finally, the cells were washed once more and then run on a flow cytometer (see., e.g., FIGS. 8A and 9).

### Example 7: Biotin Cage Staining

A single cell suspension (2×10⁷ cells) of splenocytes was incubated with 1.25 decoy tetramer (Biotin Cage::UIS4 Green) in FACs buffer for 10 minutes at room temperature. Next, the cells were incubated the cells with 1.25 μg Biotin Cage: :MSP1-Red in FACs buffer (PBS+2% FCS+2 mM EDTA) on ice for 30 minutes (no wash between). Cells were then washed cells twice with FACs buffer and centrifuged at 1600 rpm for 8 minutes. Cells were next labelled with 20 μL streptavidin- microbeads (from miltenyi Biotec) and incubated for 15 minutes in a refrigerator. Cells were then washed with 2 mL FACs buffer and centrifuged at 1600 rpm for 10 minutes. Supernatant was then aspirated and the cells were resuspended in 500 μL MACs (PBS+0.5% BSA+2 mM EDTA) buffer.

The assembly then proceeded to magnetic separation by first placing a MACs LS column in a magnetic field and washing the column with 3 mL of MACs buffer. The cell suspension was applied onto the column and unlabeled cells that pass through were collected in a new tube. The column was then washed 3 mL of MACs buffer and then removed from the magnetic field and placed on a new collection tube. 3 mL of MACs buffer was then added onto the column and the magnetically labeled cells were flushed out by firmly pushing a plunger into the column. The labeled cells (MSP1-postive cells) were washed and a cell count was obtained.

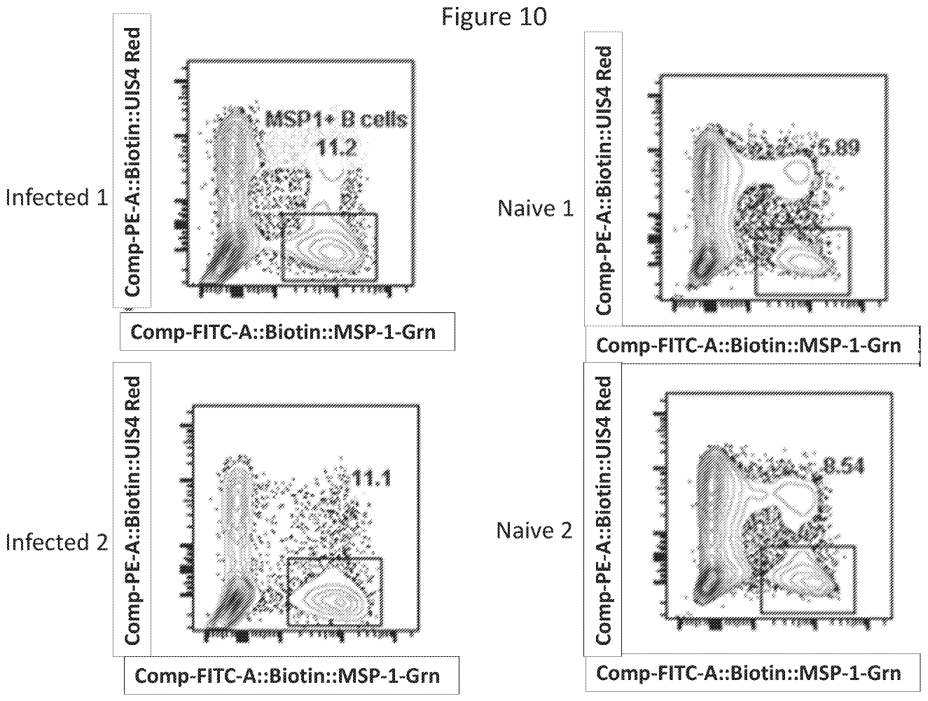

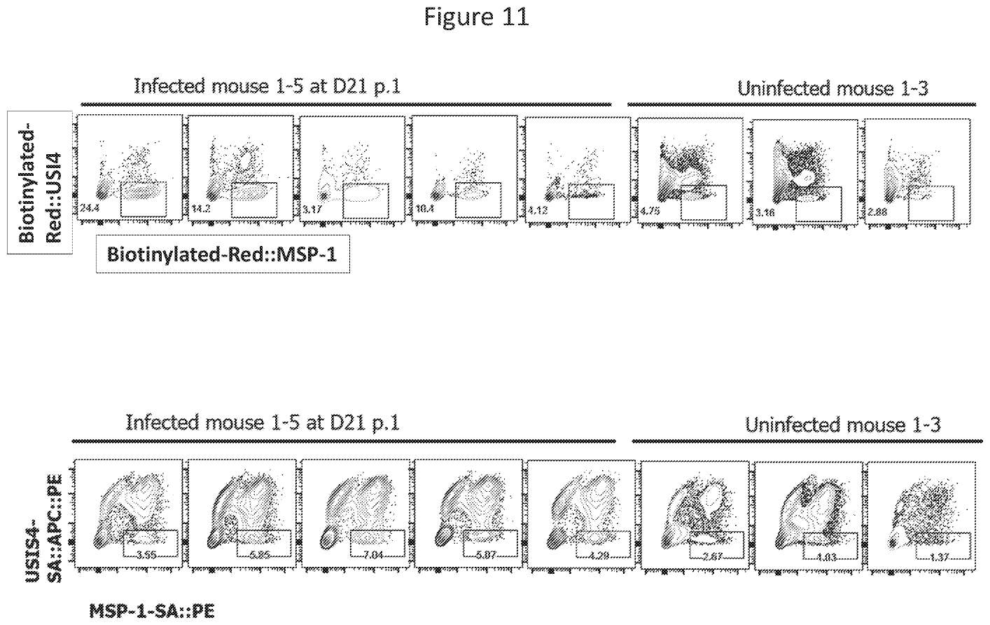

The cells were next stained with Zombie NIR dye (1:1000 dilution in PBS) at room temperature for 20 minutes and then washed once with FACs buffer. Cells were next stained with CD19 and B220 in FACs buffer on ice for 30 minutes. The final stained cells were then washed and run through a flow cytometer (see, e.g., FIGS. 10 and 11).

As depicted in FIG. 7A, the biotinylated variants can be directed incubated with a streptavidin magnetic bead. As with the procedures described above, mice were infected with P. yoelii and isolated spleen cells were allowed to interact with the assembled complexes, using the UIS4 decoy first, followed by the MSP1(19). FIG. 10 shows resulting FACS data obtained from inoculated and naive mice, showing the system isolates antigen specific B cells.

The biotinylated variants were also compared to the biotin-streptavidin tetramer model. FIG. 11 shows that the biotinylated complex (no Ab-biotinylation) offered better isolation of antigen specific B cells than that seen with the standard tetramer model.

### Example 8: Identification of B cells Responsive to SARS-CoV-2 Spike Protein

The nucleotide sequence of the spike protein from the virus SAR-CoV-2 is obtained from NCBI and then modified to improve solubility and to further include a capture sequence (SEQ ID NO: 26) and a histidine octamer. The resulting nucleotide sequence is set forth in SEQ ID NO: 33 and the coded amino acid sequence is set forth in SEQ ID NO: 32. The production of the multimeric protein structure and the capture tag target protein follow the same procedures as described herein, differing only in the expressed target protein. Importantly, the Capture-Tag sequence at the C-terminus provides for specific covalent binding to the biotinylated or unbiotinylated multimer protein structure variants as described herein. Populations of B cells from infected subjects either exposed to or suspected of being exposed to SARS-CoV-2 are then allowed to incubate with the either the generated SARS-CoV-2 Capture-Cage or Biotin Cage constructs, followed by magnetic isolation of responsive B cells and/or T cells to the affixed SARS-CoV-2 antigen and then optional flow cytometry. B cells can then proliferate in vitro or be further processed for RNA isolation to identify the sequences of antibodies specific to binding the SARS-CoV-2 antigen and generate recombinant antibodies or the relevant CDRs for specific binding.

### Example 10: Identification of B Cells Responsive to HA protein of influenza H1N1 or MSP1 of Plasmodium falciparum

As with Example 9, different target proteins are introduced into the multimer complex. The cDNA sequence for a modified HA of H1N1 is set forth in SEQ ID NO: 35 and the modified MSP1(19) from P. falciparum is set forth in SEQ ID NO: 37. The corresponding amino acid sequences for each are set forth in SEQ ID NOs: 34 and 36, respectively. As with Example 9, incubation with cells comprising adaptive immune cells allows for isolation of B and/or T cells that recognize the respective target protein, thereby allowing for establishing cell cultures and/or the isolation of antibodies or relevant fragments thereof pertinent to binding specificity.

The foregoing description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Various modifications of the present invention, in addition to those shown and described herein, will be apparent to those skilled in the art of the above description.

It is appreciated that all reagents used in the manufacture or use of the materials of the present disclosure are obtainable by sources known in the art unless otherwise specified.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

1. A method for isolating cells responsive to a target protein comprising:
(a) contacting a collection of isolated cells in an in vitro sample to a complex, the complex comprising a target protein with a capture tag coupled to a multimeric protein structure of at least two self-assembled copies of a monomeric protein substructure fused with a capture sequence and optionally a linker and incubating therewith; and,
(b) isolating the complex.

2. The method of claim 1, wherein the monomeric protein substructure is further fused with a complementary affinity sequence.

3. The method of claim 2, wherein the complementary affinity sequence is a biotin tag.

4. The method of claim 2, wherein step (b) is performed by introducing beads affixed with the complementary binding partner to the complementary affinity sequence and isolating the beads.

5. The method of claim 4, wherein the complementary binding partner is avidin or streptavidin.

6. The method of claim 1, further comprising before (b) incubating the complex with an antibody, wherein the antibody is biotinylated and binds to the monomeric protein substructure.

7. The method of claim 6, wherein step (b) is performed by introducing beads affixed with avidin to the in vitro solution and isolating the beads.

8. The method of claim 4, wherein the beads are magnetic.

9. (canceled)

10. The method of claim 1, wherein the monomeric protein substructure has at least 85% sequence identity with an amino acid selected from the group consisting of SEQ ID NO: 1, SEQ ID NO: 2 or SEQ ID NO: 3.

11. The method of claim 1, wherein the capture sequence has at least 85% sequence identity with an amino acid selected from the group consisting of SEQ ID NO: 7, SEQ ID NO: 8 or SEQ ID NO: 9.

12. The method of claim 1, wherein the monomeric protein substructure is further fused with a fluorophore.

13. The method of claim 12, wherein the monomeric protein substructure has at least 85% sequence identity with an amino acid selected from the group consisting of SEQ ID NO: 16, SEQ ID NO: 17, SEQ ID NO: 18 or SEQ ID NO: 19.

14. The method of claim 1, wherein the capture tag has at least 90% sequence identity with an amino acid selected from the group consisting of SEQ ID NO: 26 or SEQ ID NO: 27.

15. The method of claim 4, further comprising isolating cells bound to the complex by flow cytometry.

16. The method of claim 1, wherein the collection of cells comprise adaptive immune cells.

17. (canceled)

18. (canceled)

19. The method of claim 1, further comprising isolating nucleic acids from cells associated with the complex in (b).

20. The method of claim 1, further comprising isolating a nucleic acid encoding an antibody after (b).

21. (canceled)

22. The method of claim 1, wherein the sample further comprises a second complex, the second complex featuring a second target protein different from the first.

23. (canceled)

24. A method for assaying a subject for immunity to a target protein comprising:
(a) incubating a collection of cells isolated from the subject in an in vitro solution with a complex, the complex comprising a target protein with a capture tag coupled to a multimeric protein structure of at least two self-assembled copies of a monomeric protein substructure fused with a capture sequence and a linker and incubating therewith; and,
(b) measuring the complex and analyzing for associated proteins.

25. A method for preparing a B cell in vitro tissue culture with binding affinity to a target protein comprising:
(a) incubating a collection of cells comprised of B cells in an in vitro solution with a complex, the complex comprising a target protein with a capture tag coupled to a multimeric protein structure of at least two self-assembled copies of a monomeric protein substructure fused with a capture sequence and a linker and incubating therewith;
(b) isolating the complex;
(c) isolating B cells from the complex; and
(d) transferring isolated B cells from (c) to a tissue culture medium.

26.-51. (canceled)


SEQUENCE LISTING (38 sequences)

SEQ ID NO: 1 - protein, 205 aa, Artificial Sequence
  Synthetic Construct
MEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 2 - protein, 205 aa, Artificial Sequence; identical to SEQ ID NO: 1
  Synthetic Construct

SEQ ID NO: 3 - protein, 201 aa, Artificial Sequence
  Synthetic Construct
FKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 4 - protein, 207 aa, Artificial Sequence
  Synthetic Construct
MKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 5 - protein, 207 aa, Artificial Sequence
  Synthetic Construct
ASMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 6 - protein, 204 aa, Artificial Sequence
  Synthetic Construct
EELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 7 - protein, 98 aa, Artificial Sequence
  Synthetic Construct
GSGDSATHIKFSKRDEDGKELAGATMELRDSSGKTISTWISDGQVKDFYLYPGKYTFVETAAPDGYEVATAITFTVNEQGQVTVNGKATKGDAHIGVD

SEQ ID NO: 8 - protein, 108 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVD

SEQ ID NO: 9 - protein, 113 aa, Artificial Sequence
  Synthetic Construct
MKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 10 - protein, 5 aa, Artificial Sequence
  Synthetic Construct
EAAAK

SEQ ID NO: 11 - protein, 333 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDHHHHHHGGSGGSGGSGGSMKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 12 - protein, 340 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDEAAAKEAAAKEAAAKEAAAKEAAAKASMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 13 - protein, 337 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDEAAAKEAAAKEAAAKEAAAKEAAAKEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 14 - protein, 321 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDPPPPPPPPPEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 15 - protein, 321 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDPPAPPAPPAEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHM

SEQ ID NO: 16 - protein, 576 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDHHHHHHGGSGGSGGSGGSMKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHMGGSGGSGGSGGSVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 17 - protein, 581 aa, Artificial Sequence
  Synthetic Construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDHHHHHHGGSGGSGGSGGSMKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHMGGSGGSGGSGGSMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 18 - protein, 591 aa, Artificial Sequence
  Synthetic Construct
MGLNDIFEAQKIEWHEGGSGGSGGSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDGGSGGSGGSGGSMKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHMGGSGGSGGSGGSVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 19 - protein, 596 aa, Artificial Sequence
  Synthetic Construct
MGLNDIFEAQKIEWHEGGSGGSGGSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDGGSGGSGGSGGSMKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHMGGSGGSGGSGGSMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 20 - protein, 599 aa, Artificial Sequence
  Synthetic Construct
MGLNDIFEAQKIEWHEGGSGGSGGSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGVDGGSGGSGGSGGSMKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEHMGGSGGSGGSGGSMVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 21 - DNA, 1731 bp, Artificial Sequence
  Synthetic Construct
atgggcagcagccatcatcatcatcatcacggcagcggcgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaacggcaaagcaactaaaggtgacgctcatattggcgtcgaccaccaccaccaccaccacggcggcagcggcggcagcggcggtagcggcggtagcatgaagatggaagagctgttcaagaaacacaagatcgttgccgtgctgcgtgccaatagtgtggaagaagcgaaaaagaaagcgctggcggttttcctgggcggcgttcatctgattgaaattacctttaccgtgccggatgcggataccgtgattaaggaactgagctttctgaaggaaatgggcgcgattattggtgcgggcaccgtgaccagcgtggagcagtgccgtaaagcggtggaaagtggcgccgaattcattgtgagtccgcacctggacgaggaaattagccaattttgcaaggagaagggtgtgttctatatgccaggcgttatgaccccgaccgaactggtgaaagccatgaaactgggccataccatcttaaaactgtttccgggtgaggtggtgggtccgcagtttgttaaagcgatgaaaggtccgtttccgaatgtgaaatttgtgccaaccggcggtgttaatctggacaatgtgtgcgaatggttcaaagcgggcgtgctggccgtgggcgtgggcagcgcgttagtgaaaggcaccccggtggaagtggcggaaaaggccaaggcgttcgttgagaagattcgtggctgcaccgaacatatgggtggcagcggaggctctggaggttccggcggatctgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagcttcgcggcaccaacttccctcctgacggccccgtaatgcagaagaagacaatgggctgggaagcatccaccgagcggttgtaccccgaggacggcgtgctgaagggcgacattaagatggccctgcgcctgaaggacggcggtcgctacctggcggacttcaagaccacctacaaggccaagaagcccgtgcagatgcccggcgcctacaacgtcgatcgcaagttggacatcacctcccacaacgaggactacaccgtggtggaacagtacgaacgctccgagggccgccactccaccggcggcatggacgagctgtacaagtaa

SEQ ID NO: 22 - DNA, 1746 bp, Artificial Sequence
  Synthetic Construct
atgggcagcagccatcatcatcatcatcacggcagcggcgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaacggcaaagcaactaaaggtgacgctcatattggcgtcgaccaccaccaccaccaccacggcggcagcggcggcagcggcggtagcggcggtagcatgaagatggaagagctgttcaagaaacacaagatcgttgccgtgctgcgtgccaatagtgtggaagaagcgaaaaagaaagcgctggcggttttcctgggcggcgttcatctgattgaaattacctttaccgtgccggatgcggataccgtgattaaggaactgagctttctgaaggaaatgggcgcgattattggtgcgggcaccgtgaccagcgtggagcagtgccgtaaagcggtggaaagtggcgccgaattcattgtgagtccgcacctggacgaggaaattagccaattttgcaaggagaagggtgtgttctatatgccaggcgttatgaccccgaccgaactggtgaaagccatgaaactgggccataccatcttaaaactgtttccgggtgaggtggtgggtccgcagtttgttaaagcgatgaaaggtccgtttccgaatgtgaaatttgtgccaaccggcggtgttaatctggacaatgtgtgcgaatggttcaaagcgggcgtgctggccgtgggcgtgggcagcgcgttagtgaaaggcaccccggtggaagtggcggaaaaggccaaggcgttcgttgagaagattcgtggctgcaccgaacatatgggtggcagcggaggctctggaggttccggcggatctatggtgtcgaagggggaagaggataacatggctagtcttccagcgacacacgagcttcacattttcggttctatcaatggagtggatttcgacatggttggccaaggaacaggcaaccctaatgatggatatgaagaacttaatcttaaatctactaaaggagacctgcaattcagcccctggattctggtccctcacattgggtacggttttcaccagtatcttccatatccggacggtatgtctcctttccaagcggctatggtggacggctcgggctatcaagtccatcgtaccatgcagtttgaagatggcgcgtcactgactgtgaattaccgttacacatacgagggtagtcatatcaagggagaggcccaagtcaagggaacgggttttcccgccgatgggccagtaatgacaaattctcttaccgctgccgattggtgtcgtagtaaaaaaacatacccaaacgataagaccattatctcaacgttcaagtggagttacacaaccgggaacggaaagcgctaccgttccaccgcacgcacgacttacacgttcgcgaagccaatggccgctaattacctgaaaaatcagcctatgtacgtcttccgtaagactgagttaaagcacagtaagacagagctgaacttcaaggaatggcagaaggcgtttacagacgtaatgggtatggatgagttgtataagtag

SEQ ID NO: 23 - DNA, 1776 bp, Artificial Sequence
  Synthetic Construct
atgggcctaaatgatatctttgaagcacagaaaatcgaatggcacgaaggtgggagcgggggctcgggcggaagtcaccatcatcaccatcacggcagcggcgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaacggcaaagcaactaaaggtgacgctcatattggcgtcgacggtggcagcggcgggagtggaggttctggtgggtcaatgaagatggaagagctgttcaagaaacacaagatcgttgccgtgctgcgtgccaatagtgtggaagaagcgaaaaagaaagcgctggcggttttcctgggcggcgttcatctgattgaaattacctttaccgtgccggatgcggataccgtgattaaggaactgagctttctgaaggaaatgggcgcgattattggtgcgggcaccgtgaccagcgtggagcagtgccgtaaagcggtggaaagtggcgccgaattcattgtgagtccgcacctggacgaggaaattagccaattttgcaaggagaagggtgtgttctatatgccaggcgttatgaccccgaccgaactggtgaaagccatgaaactgggccataccatcttaaaactgtttccgggtgaggtggtgggtccgcagtttgttaaagcgatgaaaggtccgtttccgaatgtgaaatttgtgccaaccggcggtgttaatctggacaatgtgtgcgaatggttcaaagcgggcgtgctggccgtgggcgtgggcagcgcgttagtgaaaggcaccccggtggaagtggcggaaaaggccaaggcgttcgttgagaagattcgtggctgcaccgaacatatgggtggcagcggaggctctggaggttccggcggatctgtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagcttcgcggcaccaacttccctcctgacggccccgtaatgcagaagaagacaatgggctgggaagcatccaccgagcggttgtaccccgaggacggcgtgctgaagggcgacattaagatggccctgcgcctgaaggacggcggtcgctacctggcggacttcaagaccacctacaaggccaagaagcccgtgcagatgcccggcgcctacaacgtcgatcgcaagttggacatcacctcccacaacgaggactacaccgtggtggaacagtacgaacgctccgagggccgccactccaccggcggcatggacgagctgtacaagtaa

SEQ ID NO: 24 - DNA, 1791 bp, Artificial Sequence
  Synthetic Construct
atgggcctaaatgatatctttgaagcacagaaaatcgaatggcacgaaggtgggagcgggggctcgggcggaagtcaccatcatcaccatcacggcagcggcgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaacggcaaagcaactaaaggtgacgctcatattggcgtcgacggtggcagcggcgggagtggaggttctggtgggtcaatgaagatggaagagctgttcaagaaacacaagatcgttgccgtgctgcgtgccaatagtgtggaagaagcgaaaaagaaagcgctggcggttttcctgggcggcgttcatctgattgaaattacctttaccgtgccggatgcggataccgtgattaaggaactgagctttctgaaggaaatgggcgcgattattggtgcgggcaccgtgaccagcgtggagcagtgccgtaaagcggtggaaagtggcgccgaattcattgtgagtccgcacctggacgaggaaattagccaattttgcaaggagaagggtgtgttctatatgccaggcgttatgaccccgaccgaactggtgaaagccatgaaactgggccataccatcttaaaactgtttccgggtgaggtggtgggtccgcagtttgttaaagcgatgaaaggtccgtttccgaatgtgaaatttgtgccaaccggcggtgttaatctggacaatgtgtgcgaatggttcaaagcgggcgtgctggccgtgggcgtgggcagcgcgttagtgaaaggcaccccggtggaagtggcggaaaaggccaaggcgttcgttgagaagattcgtggctgcaccgaacatatgggtggcagcggaggctctggaggttccggcggatctatggtgtcgaagggggaagaggataacatggctagtcttccagcgacacacgagcttcacattttcggttctatcaatggagtggatttcgacatggttggccaaggaacaggcaaccctaatgatggatatgaagaacttaatcttaaatctactaaaggagacctgcaattcagcccctggattctggtccctcacattgggtacggttttcaccagtatcttccatatccggacggtatgtctcctttccaagcggctatggtggacggctcgggctatcaagtccatcgtaccatgcagtttgaagatggcgcgtcactgactgtgaattaccgttacacatacgagggtagtcatatcaagggagaggcccaagtcaagggaacgggttttcccgccgatgggccagtaatgacaaattctcttaccgctgccgattggtgtcgtagtaaaaaaacatacccaaacgataagaccattatctcaacgttcaagtggagttacacaaccgggaacggaaagcgctaccgttccaccgcacgcacgacttacacgttcgcgaagccaatggccgctaattacctgaaaaatcagcctatgtacgtcttccgtaagactgagttaaagcacagtaagacagagctgaacttcaaggaatggcagaaggcgtttacagacgtaatgggtatggatgagttgtataagtag

SEQ ID NO: 25 - DNA, 1800 bp, Artificial Sequence
  Synthetic Construct
atgggcctaaatgatatctttgaagcacagaaaatcgaatggcacgaaggtgggagcgggggctcgggcggaagtcaccatcatcaccatcacggcagcggcgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaacggcaaagcaactaaaggtgacgctcatattggcgtcgacggtggcagcggcgggagtggaggttctggtgggtcaatgaagatggaagagctgttcaagaaacacaagatcgttgccgtgctgcgtgccaatagtgtggaagaagcgaaaaagaaagcgctggcggttttcctgggcggcgttcatctgattgaaattacctttaccgtgccggatgcggataccgtgattaaggaactgagctttctgaaggaaatgggcgcgattattggtgcgggcaccgtgaccagcgtggagcagtgccgtaaagcggtggaaagtggcgccgaattcattgtgagtccgcacctggacgaggaaattagccaattttgcaaggagaagggtgtgttctatatgccaggcgttatgaccccgaccgaactggtgaaagccatgaaactgggccataccatcttaaaactgtttccgggtgaggtggtgggtccgcagtttgttaaagcgatgaaaggtccgtttccgaatgtgaaatttgtgccaaccggcggtgttaatctggacaatgtgtgcgaatggttcaaagcgggcgtgctggccgtgggcgtgggcagcgcgttagtgaaaggcaccccggtggaagtggcggaaaaggccaaggcgttcgttgagaagattcgtggctgcaccgaacatatgggtggcagcggaggctctggaggttccggcggatctatggtaagcaagggagaagaactgtttacaggagttgttcctatcttagttgaacttgacggcgacgttaacggccacaagttttccgtgagcggagagggtgagggcgatgccacttacggtaaattgactttaaaattcatctgcactaccggcaaacttcccgttccgtggcccaccttggtaaccaccctttcctggggggtccagtgctttgcacgctatccagatcacatgaagcaacacgatttttttaagagtgcaatgccggaaggttatgtccaagagcgcactatcttttttaaggatgacggaaattacaagactcgcgcggaagtgaagtttgagggagacacccttgttaaccgcattgaattgaagggcatcgacttcaaggaggatggaaacatcttagggcataaacttgagtataactatttttcagataatgtatatatcacagctgataaacaaaagaatggcatcaaagcgaattttaaaatccgccataacattgaggacggaggagtgcagttagcagatcattaccaacaaaacaccccgattggtgacggccctgtacttttgccagacaatcactatttgagcacccaaagtaaattgtcgaaagaccctaacgaaaagcgtgatcacatggtcttactggaatttgtcacagctgcggggatcacattaggtatggatgaactgtataagtaa

SEQ ID NO: 26 - protein, 13 aa, Artificial Sequence
  Synthetic Construct
AHIVMVDAYKPTK

SEQ ID NO: 27 - protein, 13 aa, Artificial Sequence
  Synthetic Construct
KLGDIEFIKVNKG

SEQ ID NO: 28 - protein, 402 aa, Artificial Sequence
  Synthetic Construct
MTMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSSMGMHIASIALNNLNKSGLVGEGESKKILAKMLNMDGMDLLGVDPKHVCVDTRDIPKNAGCFRDDNGTEEWRCLLGYKKGEGNTCVENNNPTCDINNGGCDPTASCQNAESTENSKKIICTCKEPTPNAYYEGVFCSSSSTSSGAHIVMVDAYKPTKGLENLYFQGVEHHHHHH

SEQ ID NO: 29 - DNA, 1209 bp, Artificial Sequence
  Synthetic Construct
atgaccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatcttccatggggatgcatattgcgtcaattgcattgaataacttaaacaaatctggcttagtcggagaaggggagtcgaaaaaaattttggcaaaaatgttaaacatggatggaatggatttacttggcgtcgatccaaagcacgtttgcgttgatacgcgcgatattcctaaaaatgcaggctgttttcgtgacgataatggtaccgaagaatggcgttgtcttcttggatacaagaaaggtgaagggaatacctgcgtagagaacaataatcccacttgcgatatcaataacggcgggtgtgacccaaccgcctcttgccaaaacgccgagtcaacggagaactctaagaagatcatttgcacctgcaaagaaccgacaccaaatgcctattatgagggggtcttctgttcttcgtcatccactagttcaggcgcccacatcgtgatggtggacgcctacaagccgacgaagggtctcgagaacctgtacttccagggagtcgagcaccaccaccaccaccactga

SEQ ID NO: 30 - protein, 410 aa, Artificial Sequence
  Synthetic Construct
MTMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSSMGSSHHHHHHSSGLVPRGSHMVREKFGIRKRIKNFDDVNTPQDISLISPVENPYQEYYPEDYQEQYPEISSDQYIEQPQKHYTKRFLEQYTNSVQNDHTYSYSPTEEKYNTYYMAPDTHDEYEKLFTDDQKEEINDNIVYHDELSDLMGEGHKIYSMNDKPFDPYIAHIVMVDAYKPTKVD

SEQ ID NO: 31 - DNA, 1233 bp, Artificial Sequence
  Synthetic Construct
atgaccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatcttccatgggcagcagccatcatcatcatcatcacagcagcggcctggtgccgcgcggcagccatatggtgcgtgaaaaatttggtattcgcaaacgtattaaaaatttcgatgacgtgaacaccccgcaggacattagcctgattagcccggtggagaatccgtaccaggaatattacccggaggactaccaggagcagtatccggagattagcagcgaccagtacatcgaacagccgcagaagcattacaccaaacgcttcctggagcagtataccaacagcgtgcagaacgatcacacctatagctacagcccgaccgaggagaagtacaacacctactacatggccccggatacccacgacgagtacgagaaactgttcaccgatgaccagaaagaagaaattaatgataatattgtgtatcatgatgaactgagtgacctgatgggcgagggccataaaatctacagcatgaatgataaaccgtttgatccgtacattgcacacatcgttatggtagatgcatataaaccaactaaagtcgactaa

SEQ ID NO: 32 - protein, 1281 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHGGGSGGGGSGGAHIVMVDAYKPTK

SEQ ID NO: 33 - DNA, 3846 bp, Artificial Sequence
  Synthetic Construct
atgtttgtttttttagtcctgctgcctctggtgtccagtcagtgcgtgaacctgaccaccaggactcagctcccccctgcatatactaacagcttcacacgcggagtgtactacccggacaaggtttttcgaagttccgtgttgcactctacacaggacctctttctcccctttttctcaaacgtcacgtggtttcatgcaatacatgtttccggaacaaacggtaccaaacgctttgataacccagtactcccttttaacgacggtgtctattttgcttctacggaaaagagcaatatcatccgtggctggatcttcggcacaaccctggactctaaaactcaaagcctcctgattgtgaataacgccacgaacgtagtgatcaaggtgtgtgagttccagttttgtaacgatccttttctgggtgtgtattaccataaaaataacaagagctggatggaatccgagtttagagtgtactcaagtgccaacaactgcacctttgaatatgttagccagccttttctgatggacctggagggaaaacagggcaactttaaaaacctcagagagttcgttttcaaaaacattgacggctatttcaagatctactctaagcacactcccattaacttggtgagggacctgccacaaggtttcagcgctctggagcccctggttgacctccccataggtattaacattacacggtttcaaacactcctggctctccatcgatcatatcttactcccggcgattcaagctcaggctggactgccggagccgctgcttactatgtaggctaccttcagcctcggacatttctcctgaaatacaatgagaacggtaccattacagatgcagtcgattgtgcccttgatccactgagtgagacaaagtgcactctcaaatccttcacggtggaaaaaggcatctaccagacctccaacttcagagtccagcccacagaaagcatcgtgcgttttccaaacatcactaacctctgtccatttggcgaggtgttcaacgcaacccggtttgccagcgtgtacgcttggaacaggaaacgaatcagcaattgtgtggccgactatagcgtcttgtataattctgcgtctttctctacatttaaatgttatggtgtatcccccacaaaactgaacgatttgtgtttcactaatgtctacgctgacagctttgtcatccgcggcgatgaggtgcgccagatcgctccagggcaaacaggtaagatagctgactataattataagcttccagacgacttcacgggatgcgtcattgcatggaatagcaacaatctcgactccaaggtggggggaaattacaactatttgtacaggctttttcgaaagtcaaatttaaaacctttcgagcgtgacatctcaaccgagatctaccaggcgggttccactccctgcaatggcgtcgagggctttaactgttacttcccccttcagagctatgggtttcaaccgacgaacggggtgggctatcaaccgtacagggtggtggtgttaagttttgaacttctgcacgcacctgccactgtctgcggcccgaaaaagtctacaaacttggttaagaacaagtgtgtcaactttaatttcaatggcctcacaggcactggtgtgctgacagaaagcaataaaaagtttctcccgtttcaacaattcgggcgagatattgcagatacaaccgatgccgtcagggatccccaaacgttagagatattggatattactccttgctcctttggtggagtctccgtaataacccctggcactaacacgtccaatcaggttgccgtcctttatcaagatgtaaactgcacagaggtaccagtcgccatccatgccgatcagctgacccctacctggcgagtgtacagcactggctccaacgtttttcagactcgcgcaggatgcttgatcggcgctgagcacgtgaacaatagctatgagtgcgacattcccatcggcgcgggcatttgtgcctcctaccaaacacaaacaaacagccctggaagcgcctcctctgtcgcctctcaaagtataattgcctatacaatgagcctgggagcagagaactcagtggcatacagcaataatagtatcgcaatacccactaactttacgatttctgttactacagaaatcctgccagtcagtatgacgaagacaagcgtagactgtacgatgtacatctgtggcgacagcactgaatgctcaaacttactgctccaatacggcagcttctgtacccagttgaatagggccttaaccggaatagccgtggagcaggataagaacactcaggaggtattcgcgcaggtgaaacagatttacaagactccacccattaaggatttcgggggattcaacttctcacagatcttacctgacccgagcaaaccatctaagagatcatttattgaggacctcctgtttaataaagtaacgttagctgacgctgggttcataaaacaatacggtgactgcctcggggacatcgccgccagagatctgatatgtgcccagaagtttaacggtctcacagtcctcccaccacttctcactgacgaaatgattgcccagtacactagcgctttactggctggaaccatcactagcggatggacattcggggcaggcgctgcactgcagataccgttcgctatgcagatggcataccgcttcaatggaatcggcgtgactcagaacgtgttatacgagaatcagaaacttatagctaaccagttcaactctgcgatcggaaaaatccaggacagtctgagcagtactgcctcagctctggggaaattgcaggacgtggtgaaccagaacgcacaggccctgaacaccttggtgaaacagctctctagtaattttggcgcgattagtagtgtcctgaacgatattctcagtaggttggacccacctgaagcagaagtgcagatcgatcggcttataaccggaagactgcagtctcttcagacttacgtgacacagcagttaatacgggccgcagagattagggccagcgcgaacctggctgctacgaaaatgtcagagtgtgtgttggggcagtccaagagagtggatttctgtggaaagggataccacctgatgagttttcctcaatcagctccacacggggtcgtcttccttcacgttacctatgttcctgctcaggagaagaatttcaccactgcaccagcgatatgtcacgatggaaaggctcactttccacgggaaggcgtgtttgtgagtaacgggacccattggttcgtgacccagagaaatttttatgagccccagatcataactacggataacacgttcgtatcaggcaactgtgacgtggtcataggcattgtgaataataccgtctatgaccccttacagccggagctggactcattcaaagaggagctggataagtattttaaaaaccacacatcacccgacgtcgacctgggcgatatcagcggtattaatgcttcagtcgtaaatatccagaaggaaatcgataggttaaacgaggtggccaaaaatctgaacgaaagcctcattgatctccaggagttggggaagtatgagcagggtagtggttacattccagaggcacccagggacggacaagcctatgttaggaaggacggcgagtgggtgttgctctctacctttcttggcaggagtctggaggtcttattccagggtcccggacaccatcatcaccaccaccatcacggcggggggagcggaggaggcggttccggtggagcacatattgtgatggttgacgcttacaagccaaccaaatag

SEQ ID NO: 34 - protein, 603 aa, Artificial Sequence
  Synthetic Construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHESNKGVTAACPHAGAKSFYKNLIWLVKKGNSYPKLSKSYINDKGKEVLVLWGIHHPPTSADQQSLYQNEDTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQGGGGGGSSSSSSSSSGYIPEAPRDGQAYVRKDGEWVLLSTFLGGSHHHHHHGGSGGSGGSAHIVMVDAYKPTKG

SEQ ID NO: 35 - DNA, 1812 bp, Artificial Sequence
  Synthetic Construct
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgaatcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccacctactagtgctgaccaacaaagtctctatcagaatgaagatacatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtgaaaagcacaaaattgagactggccacaggattgaggaatatcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagggaggtggcggtggaggcagctcctctagttcaagcagttcttccgggtacatacctgaagcgccacgagacggacaggcgtatgtgcgcaaggacggagagtgggtactcctgtctacgtttctcggcggaagccatcatcaccatcaccacggaggatctggtgggagtgggggctctgctcatattgtcatggtagatgcctataagccaactaaaggctag

SEQ ID NO: 36 - protein, 364 aa, Artificial Sequence
  Synthetic Construct
MAMTMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSSVGMNISQHQCVKKQCPENSGCFRHLDEREECKCLLNYKQEGDKCVENPNPTCNENNGGCDADATCTEEDSGSSRKKITCECTKPDSYPLFDGIFCSSSNTSSGAHIVMVDAYKPTKGLENLYFQGLEHHHHHH

SEQ ID NO: 37 - DNA, 1095 bp, Artificial Sequence
  Synthetic Construct
atggccatgaccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatcttccgtggggatgaacatctctcagcatcagtgtgttaaaaagcaatgtcctgagaactccgggtgtttccgccacttggatgaacgtgaagagtgtaagtgtttgctgaactataagcaagagggagacaagtgtgttgagaatcctaacccaacatgtaacgaaaataacggcgggtgtgacgcagacgcgacgtgtactgaggaagatagcgggtccagtcgcaaaaagatcacttgcgaatgcacaaaacccgacagctacccactttttgatggaatcttttgcagctcatcaaatactagttcaggcgcccacatcgtgatggtggacgcctacaagccgacgaagggtctcgagaacctgtacttccagggactcgagcaccaccaccaccaccactga

SEQ ID NO: 38 - protein, 15 aa, Artificial Sequence
  Synthetic Construct
GLNDIFEAQKIEWHE